SEQUENCE LISTING (85 sequences)

SEQ ID NO: 1 - protein, 104 aa, Homo sapiens
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKR

SEQ ID NO: 2 - protein, 49 aa, Homo sapiens
QVQLVQSGGQMKKPGESMRISCRASGYEFIDCTLNWIRLAPGKRPEWMG

SEQ ID NO: 3 - protein, 104 aa, Homo sapiens
EIVLTQSPGTLSLSPGETAIISCRTSQYGSLAWYQQRPGQAPRLVIYSGSTRAAGIPDRFSGSRWGPDYNLTIRNLESGDFGLYYCQQYEFFGQGTKVQVDIKR

SEQ ID NO: 4 - protein, 121 aa, Homo sapiens
QVQLVQSGGQMKKPGESMRISCQASGYEFIDCTLNWVRLAPGRRPEWMGWLKPRGGAVNYARPLQGRVTMTRDVYSDTAFLELRSLTADDTAVYYCTRGKNCDYNWDFEHWGRGTPVTVSS

SEQ ID NO: 5 - protein, 107 aa, Homo sapiens
EIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKRTVA

SEQ ID NO: 6 - protein, 125 aa, Homo sapiens
QVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSS

SEQ ID NO: 7 - protein, 103 aa, Homo sapiens
DIQMTQSPSSLSARVGDTVTITCQANGYLNWYQQRRGKAPKLLIYDGSKLERGVPARFSGRRWGQEYNLTINNLQPEDVATYFCQVYEFIVPGTRLDLKRTVA

SEQ ID NO: 8 - protein, 123 aa, Homo sapiens
QVHLSQSGAAVTKPGASVRVSCEASGYKISDHFIHWWRQAPGQGLQWVGWINPKTGQPNNPRQFQGRVSLTRQASWDFDTYSFYMDLKAVRSDDTAIYFCARQRSDFWDFDVWGSGTQVTVSS

SEQ ID NO: 9 - protein, 103 aa, Homo sapiens
DIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLKRTVA

SEQ ID NO: 10 - protein, 123 aa, Homo sapiens
QVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSS

SEQ ID NO: 11 - protein, 103 aa, Homo sapiens
DIQMTQSPSSLSARVGDTVTITCQANGYLNWYQQRRGKAPKLLIYDGSKLETGVPSRFTGRRWGQEYNLTINNLQPEDIATYFCQVYEFIVPGTRLDLKRTVA

SEQ ID NO: 12 - protein, 123 aa, Homo sapiens
QVRLLQSGAAVTKPGASVRVSCEASGYEIRDYFIHWWRQAPGQGLQWVGWINPKTGQPNNPRQFQGRVSLTRQASWDFDSYSFYMDLKALRSDDTGVYFCARQRSDYWDFDVWGSGTQVTVSS

SEQ ID NO: 13 - protein, 103 aa, Homo sapiens
DIQMTQSPSSLSASVGDTVTITCQANGYLNWYQQRRGKAPKLLIYDGSKLERGVPSRFSGRRWGQEYNLTINNLQPEDIATYFCQVYEFIVPGTRLDLKRTVA

SEQ ID NO: 14 - protein, 123 aa, Homo sapiens
QVQLLQSGAAVTKPGASVRVSCEASGYNIRDYFIHWWRQAPGQGLQWVGWINPKTGQPNNPRLFQGRVSLTRHASWDFDTFSFYMDLKAVRSDDTAVYFCARQRSDYWDFDVWGSGTQVTVSS

SEQ ID NO: 15 - protein, 103 aa, Homo sapiens
DIQMTQSPSSLSASVGDTVTITCQANGYLNWYQQRRGKAPKLLIYDGSKLERGVPSRFSGRRWGQEYNLTINNLQAEDIATYFCQVYEFAVPGTRLDLKRTVA

SEQ ID NO: 16 - protein, 123 aa, Homo sapiens
QVQLLQSGAAVTKPGASVRVSCEASGYNIRDYFIHWWRQAPGQGLQWVGWINPKTGQPNNPRQFQGRVSLTRHASWDFDTFSFYMDLKGLRSDDTAIYFCARQRSDYWDFDVWGSGTQVTVSS

SEQ ID NO: 17 - protein, 107 aa, Homo sapiens
DIQMTQSPSSLSASVGDRVTITCQAGQGIGSSLQWYQQKPGKAPKLLVHGASNLHRGVPSRFSGSGFHTTFSLTISGLQRDDFATYFCAVLEFFGPGTKVEIKRTVA

SEQ ID NO: 18 - protein, 122 aa, Homo sapiens
SQHLVQSGTQVKKPGASVRISCQASGYSFTDYVLHWWRQAPGQGLEWMGWIKPVYGARNYARRFQGRINFDRDIYREIAFMDLSGLRSDDTALYFCARDGSGDDTSWHLDPWGQGTLVIVSA

SEQ ID NO: 19 - protein, 102 aa, Homo sapiens
EIVLTQSPGTLSLSPGETASLSCTAASYGHMTWYQKKPGQPPKLLIFATSKRASGIPDRFSGSQFGKQYTLTITRMEPEDFARYYCQQLEFFGQGTRLEIRR

SEQ ID NO: 20 - protein, 125 aa, Homo sapiens
QVQLVQSGSGVKKPGASVRVSCWTSEDIFERTELIHWVRQAPGQGLEWIGWVKTVTGAVNFGSPDFRQRVSLTRDRDLFTAHMDIRGLTQGDTATYFCARQKFYTGGQGWYFDLWGRGTLIVVSS

SEQ ID NO: 21 - protein, 103 aa, Homo sapiens
DIQMTQSPSSLSASLGDRVTITCQASRGIGKDLNWYQQKPGKAPKLLVSDASILEGGVPSRFSGSGFHQNFSLTISSLQPEDVATYFCQQYETFGQGTKVDIK

SEQ ID NO: 22 - protein, 131 aa, Homo sapiens
QVQLVQSGAAVRKPGASVTVSCKFAEDDDYSPHWVNPAPEHYIHFLRQAPGQQLEWLAWMNPTNGAVNYAWQLHGRLTATRDGSMTTAFLEVRSLRSDDTAVYYCARAQKRGRSEWAYAHWGQGTPVLVSS

SEQ ID NO: 23 - protein, 103 aa, Homo sapiens
DIQMTQSPSSLSASLGDRVTITCQASRGIGKDLNWYQQKAGKAPKLLVSDASTLEGGVPSRFSGSGFHQNFSLTISSLQAEDVATYFCQQYETFGQGTKVDIK

SEQ ID NO: 24 - protein, 131 aa, Homo sapiens
QVQLVQSGAAVRKPGASVTVSCKFAEDDDYSPYWVNPAPEHFIHFLRQAPGQQLEWLAWMNPTNGAVNYAWYLNGRVTATRDRSMTTAFLEVKSLRSDDTAVYYCARAQKRGRSEWAYAHWGQGTPVVVSS

SEQ ID NO: 25 - protein, 103 aa, Homo sapiens
DIQMTQSPSSLSASLGDRVTITCQASRGIGKDLNWYQQKPGRAPKLLVSDASILEGGVPTRFSGSGFHQNFSLTISSLQAEDVATYFCQQYETFGQGTKVDIK

SEQ ID NO: 26 - protein, 131 aa, Homo sapiens
QVQLVQSGAAVRKPGASVTVSCKFAEDDDFSPHWVNPAPEHYIHFLRQAPGQQLEWLAWMKPTNGAVNYAWQLQGRVTVTRDRSQTTAFLEVKNLRSDDTAVYYCARAQKRGRSEWAYAHWGQGTPVVISA

SEQ ID NO: 27 - protein, 103 aa, Homo sapiens
DIQMTQSPSSLSASLGDRVTITCQASRGIGKDLNWYQQKRGRAPRLLVSDASVLEGGVPSRFSGSGFHQNFSLTISTLQPEDVATYFCQQYETFGQGTKVDIK

SEQ ID NO: 28 - protein, 131 aa, Homo sapiens
QVQLVQSGAAVRKPGASISVSCKFADADDYSPHWMNPAPEHYIHFLRQAPGQQLEWLAWMNPTNGAVNYAWYLNGRVTATRDRSMTTAFLEVRSLRSDDTAVYYCARAQKRARSEWAYAHWGQGTPVVVSS

SEQ ID NO: 29 - protein, 103 aa, Homo sapiens
DIQMTQSPSSLSASLGDRVTITCQASRGIGKDLNWYQQKAGKAPKLLVSDASILEGGVPSRFSGSGFHQNFSLTISSLQPEDVATYFCQQYETFGQGTKVDIK

SEQ ID NO: 30 - protein, 131 aa, Homo sapiens
QVQLVQSGAAVRKPGASVTVSCKFAEDDDWSPHWVNPAPEHYIHFLRQAPGQQLEWLAWMNPTNGAVNYAWQLNGRLTATRDTSMTTAFLEVKSLRSDDTAVYYCARAQKRGRSEWAYAHWGQGTPVVVSS

SEQ ID NO: 31 - protein, 103 aa, Homo sapiens
EIVLTQSPGILSLSPGETATLFCKASQGGNAMTWYQKRRGQVPRLLIYDTSRRASGVPDRFVGSGSGTDFFLTINKLDREDFAVYYCQQFEFFGLGSELEVHR

SEQ ID NO: 32 - protein, 130 aa, Homo sapiens
QVQLVQSGAVIKTPGSSVKISCRASGYNFRDYSIHWVRLIPDKGFEWIGWIKPLWGAVSYARQLQGRVSMTRQLSQDPDDPDWGVAYMEFSGLTPADTAEYFCVRRGSCDYCGDFPWQYWGQGTVVVVSS

SEQ ID NO: 33 - protein, 104 aa, Homo sapiens
DIQMTQSPSSLSASVGDKVTITCQTSAGYLNWYQQRRGRAPKLLMYDGSRLVTGVPSRFSGRRWGTQYNLTIGSLQPEDIATYYCQVYEFFGPGTRLDLKSTVA

SEQ ID NO: 34 - protein, 119 aa, Homo sapiens
QVQLVQSGTAVKRPGASVRVSCQASGYTFTDYFIYWWRQAPGQGLEWLGWINPLTSQPSYPSRFQGRLTLTRDTFDEMLYMDLRGLRSDDTGIYFCARRHSDYCDFDIWGSGTQIIVSS

SEQ ID NO: 35 - protein, 103 aa, Homo sapiens
DIQMTQSPSSLSARVGDTVTITCQANGYLNWYQQRRGKAPKLLIYDGSKLERGVPSRFSGRRWGQEYNLTINNLQPEDIATYFCQVYEFAVPGTRLDLKRTVA

SEQ ID NO: 36 - protein, 123 aa, Homo sapiens
QVQLLQSGAVVTKPGASVRVSCEASGYKIRDYFIHWWRQAPGQGLQWVGWINPQTGQPNIPRPFQGRVTLTRHASWDFDTFSFYMDLKALRSDDTAIYFCARRRSDYCDFDVWGSGTHVTVSS

SEQ ID NO: 37 - protein, 107 aa, Homo sapiens
DIQMTQSPSSLSASIGDRVNITCQASRDTGSALNWYQQKVGRPPRLLISAVSNLGAGVPSRFSGRRSGTQSTLTINTLQPEDIATYFCQHYEFFGPGTKVDIKRTVA

SEQ ID NO: 38 - protein, 125 aa, Homo sapiens
EVQLVQSGSDVRKPGATVTVSCKADEDEDDFTAYNYFMHWVRQAPGHGLEWIGWINPRTGQPNHAKQFQGRVTLTRERSTSTVFMKLTNLRLDDTAVYFCARPLRGGDTWHYHSWGRGTSLTVSS

SEQ ID NO: 39 - protein, 104 aa, Homo sapiens
DIQMTQSPSSLSASVGDKVTITCQTSAGYLNWYQQRRGRAPKLLMYDGSRLVTGVPSRFSGRRWGTQYNLTIGSLQPEDVATYYCQVYEFFGPGTRLDLKRTVA

SEQ ID NO: 40 - protein, 119 aa, Homo sapiens
QVQLVQSGTAVKRPGASVRVSCQASGYTFIDHFIYWWRQAPGQGLEWLGWINPLTSQPSYPSRFQGRLTLTRDTFDEMLYMDLRGLRSDDTGIYFCARRHSDYCDFDIWGSGTQIIVSS

SEQ ID NO: 41 - protein, 122 aa, Homo sapiens
DIQMTQSPSSLSASVGDRVTINCQAGQGIGSSLNWYQKKPGRAPKLLVHGASNLQRGVPSRFSGSGFHTTFTLTISSLQPDDVATYFCAVFQWFGPGTKVDIKRTVAAPSVFIFPPSDEQLK

SEQ ID NO: 42 - protein, 127 aa, Homo sapiens
SQHLVQSGTQVKKPGASVRVSCQASGYTFTNYILHWWRQAPGQGLEWMGLIKPVFGAVNYARQFQGRIQLTRDIYREIAFLDLSGLRSDDTAVYYCARDESGDDLKWHLHPWGQGTQVIVSPASTKG

SEQ ID NO: 43 - protein, 101 aa, Homo sapiens
EIVLTQSPGTLSLSPGETASLSCTAASYGHMTWYQKKPGQPPKLLIFATSKRASGIPDRFSGSQFGKQYTLTITRMEPEDFAGYYCQQVEFFGQGTRLEIR

SEQ ID NO: 44 - protein, 125 aa, Homo sapiens
QVQLVQSGSGVKKPGASVRVSCWTSEDIFERTELIHWVRQAPGQGLEWIGWVKTVTGAVNFGSPNFRHRVSLTRDRDLFTAHMDIRGLTQGDTATYFCARQKFERGGQGWYFDLWGRGTLIVVSS

SEQ ID NO: 45 - protein, 128 aa, Homo sapiens
QVQLVQSGGGVKKPGTSASFSCRTSDDIYDNEFFDSAFMHWVRLIPGQRPEWMGWMNPRSGAVNYARQLQPRVSMYRDRDLSTAYMEFKSLTSADTGTYFCARKKRGDGFNLYFDLWGRGSQVIVSSA

SEQ ID NO: 46 - protein, 126 aa, Homo sapiens
QVQLVQSGSAMKKPGASVRVSCWTSEDIFDTTELIHWVRQAPGQGLEWIGWVKAVSGAVNYGSLDFRHRVSLTRDRDLSTAHMDIRGLTQDDTATYFCARQKFARGDQGWFFDLWGRGTLIVVSSA

SEQ ID NO: 47 - DNA, 22 bp, synthetic construct
  misc_feature 1..22: note = Description of Artificial Sequence: Synthetic primer
  source 1..22: mol_type = other DNA
gcctcaataaagcttgccttga

SEQ ID NO: 48 - DNA, 21 bp, synthetic construct
  misc_feature 1..21: note = Description of Artificial Sequence: Synthetic primer
  source 1..21: mol_type = other DNA
ggcgccactgctagagatttt

SEQ ID NO: 49 - DNA, 30 bp, synthetic construct
  misc_feature 1..30: note = Description of Artificial Sequence: Synthetic primer
  source 1..30: mol_type = other DNA
aagtagtgtgtgcccgtctgttrtktgact

SEQ ID NO: 50 - protein, 115 aa, Homo sapiens
QVQLVQSGAEVKKPGASVKVSCKASGYTFTGYYMHWVRQAPGQGLEWMGWINPNSGGTNYAQKFQGRVTMTRDTISSTAYMELSRLRSDDTAVYYCARYWYFDLWGRGTLVTVSS

SEQ ID NO: 51 - protein, 119 aa, Homo sapiens
QVQLVQSGAALKKPGASLRISCLTYGYKFTDHLIYWWRQAPGQGLEWIGWIKPETGQPSYSYRFQGRVSLTRDTFEEIVFMDLRGLRSDDTAIYFCARRHSDYCDFDVWGSGSQVIVSS

SEQ ID NO: 52 - protein, 119 aa, Homo sapiens
QVQLVQSGAALKKPGASLRISCQTYGYKFTDHLIYWWRQAPGQGLEWIGWIKPETGQPSYSYRFQGRVSLTRDTFEEIVFMDLRGLRSDDTAIYFCARRHSDYCDFDVWGGGSQVIVSS

SEQ ID NO: 53 - protein, 119 aa, Homo sapiens
QVQLVQSGAALKKPGASLRISCQTYGYKFTDHLIYWWRQAPGQGLEWIGWIKPETGQPSYSYRFQGRVSLTRDTFEEIVFMDLRGLRSDDTAIYFCARRHSDYCDFDVWGSGSQVLVSS

SEQ ID NO: 54 - protein, 119 aa, Homo sapiens
QVQLVQSGAALKKPGASLRISCQTYGYKFTDHLIYWWRQAPGQGLEWMGWIKPETGQPSYSYRFQGRVSLTRDTFEEIVFMDLRGLRSDDTAIYFCARRHSDYCDFDVWGSGSQVIVSS

SEQ ID NO: 55 - protein, 119 aa, Homo sapiens
QVQLVQSGAALKKPGASLRISCQTYGYKFTDHLIYWWRQAPGQGLEWIGWIKPETGQPSYSYRFQGRVSLTRDTFEEIAFMDLRGLRSDDTAIYFCARRHTDYCVFDVWGSGSQIIVSS

SEQ ID NO: 56 - protein, 119 aa, Homo sapiens
QVQLVQSGAALKKPGASVRISCQAYGYKFTDHLIYWWRQAPGQGLEWIGWIKPETGQPSYSYKFQGRVTLTRDTFEEIHFMDLRGLRYDDTATYFCARRHSDYCDFDVWGSGSQVSVSS

SEQ ID NO: 57 - protein, 119 aa, Homo sapiens
QVQLVQSGAALKKPGASVRISCQAYGYKFTDHLIYWWRQAPGQGLEWIGWIKPETGQPSYAYKFQGRVTLTRDTFEEIHFMDLRGVRNDDTATYFCARRHSDYCDFDVWGSGSQVIVSS

SEQ ID NO: 58 - protein, 119 aa, Homo sapiens
QVQLVQSGAALKKPGASVRISCQAYGYKFTDYLIHWWRQAPGQGLEWIGWIKPETGQPSYSYKFQGRVTLTRDTFEEILFMDLRGLRSDDTAIYFCARRHSDYCDFDVWGSGSQVIVSS

SEQ ID NO: 59 - protein, 127 aa, Homo sapiens
QPQLVQSGSGAEVKKPGASVRISCEASEYNVFDHFMQWVRQAPGQGLEWMGWINPRGGYPSYSPRFQGRLTFTRQPSWDDSSVTFHMELRGLRHDDTAVYYCARPHSPDDAWSLDVWGRGTLVTVSS

SEQ ID NO: 60 - protein, 123 aa, Homo sapiens
QVQLVQSGADVKKPGASVTVSCKTDEDEDDFRAHLVQWMRQAPGQRLEWVGWIKPQTGQPSYAQKFQGRVTLTREVSTSTVFLQLRNLRSDDTAVYYCARPRGGRDNWSFHVWGRGTLVTVSS

SEQ ID NO: 61 - protein, 119 aa, Homo sapiens
QVQLVQSGTAVKKPGASVRVSCQASGYTFTDYFIYWWRQAPGQGLEWLGWINPRTSQPSYPYRFQGRVTLTRDIFEEMLYMDLRGLRSDDTGIYFCARRHSDYCDFDIWGSGTQIIVSS

SEQ ID NO: 62 - protein, 127 aa, Homo sapiens
QVQLVPSGSGVKKPGASVRVSCWTSEDIFERTELIHWVRQAPGQGLEWIGWVKTVTGAVNFGSLDFRHRISLTRDRDLFTAHMDIRGLTQGDTATYFCARQKFESRYTGGQGWYFDLWGRGTHIVVS

SEQ ID NO: 63 - protein, 94 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNW

SEQ ID NO: 64 - protein, 95 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSS

SEQ ID NO: 65 - protein, 94 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNL

SEQ ID NO: 66 - protein, 104 aa, Homo sapiens
DIQMTQSPSSLSASVGDTVTITCQTNKGYLNWYQQRRGRAPKLLMYDGSKLVTGVPSRLSGRRWGTQYNLTIGSLQPEDIATYYCQVYEFFGPGTRLDLKRTVA

SEQ ID NO: 67 - protein, 104 aa, Homo sapiens
DIQMTQSPSSLSASVGDTVTITCQTNKGYLNWYQQKRGRAPKLLMYDGSKLVTGVPSRLSGRRWGTQYNLTIGSLQPEDIATYYCQVYEFFGPGTRLDLKRTVA

SEQ ID NO: 68 - protein, 104 aa, Homo sapiens; identical to SEQ ID NO: 66

SEQ ID NO: 69 - protein, 104 aa, Homo sapiens; identical to SEQ ID NO: 66

SEQ ID NO: 70 - protein, 104 aa, Homo sapiens; identical to SEQ ID NO: 66

SEQ ID NO: 71 - protein, 104 aa, Homo sapiens
DIQMTQSPSSLSASVGDTVTITCHTNKGYLNWYQQRRGRAPKLLMFDGSKLVTGVPSRFSGRRWGTQYNLTIGSLQPEDIATYYCQVYEVFGPGTRLDLKRTVA

SEQ ID NO: 72 - protein, 104 aa, Homo sapiens; identical to SEQ ID NO: 71

SEQ ID NO: 73 - protein, 104 aa, Homo sapiens
DIQMTQSPSSLSASVGDTVTITCQTTKGYLNWYQQRRGRAPKLLMFDGSKLVTGVPSRFSGRRWGTQYNLTIGSLQPEDLATYYCQVYEFFGPGTRLDLKRTVA

SEQ ID NO: 74 - protein, 107 aa, Homo sapiens
DIQMTQSPSSLSASVGDRVTITCQASQGISNSLNWYQQKPGKAPRLLIYGTSTLQRGVPSRFSGSGSGTRFTVTINSLQPEDIATYFCQHNEFFGRGTKVDIKRTVA

SEQ ID NO: 75 - protein, 108 aa, Homo sapiens
DIQMTQSPSSVSASVGDRVTITCQASRDTDNSLTWYQQKPGRPPKLLIYHVVNLGPGVPSRFSGSASSATQSTLIISDFQPDDVATYFCQNYEFFGPGTKVEIKRTVA

SEQ ID NO: 76 - protein, 103 aa, Homo sapiens
DIQMTQSPSSLSARVGDKVTITYQTSAGYLNWYQQRRGRAPKLLMYDGSRLVTGAPSRFSGRRWGTQYNLTIGSLQPEDIATYYCQVYEFFGPGTRLDLKRTV

SEQ ID NO: 77 - protein, 7 aa, synthetic construct
  REGION 1..7: note = Synthetic
  VARIANT 5: note = Xaa can be Ile or Leu
  VARIANT 6: note = Xaa can be Asn or Asp
GYEFXXC

SEQ ID NO: 78 - protein, 6 aa, synthetic construct
  REGION 1..6: note = Synthetic
KPRGGA

SEQ ID NO: 79 - protein, 12 aa, synthetic construct
  REGION 1..12: note = Synthetic
  VARIANT 3: note = Xaa can be Asn or Tyr
  VARIANT 5: note = Xaa can be Asp or Thr
GKXCXYNWDFEH

SEQ ID NO: 80 - protein, 16 aa, synthetic construct
  REGION 1..16: note = Synthetic
  VARIANT 3: note = Xaa can be Asn or Tyr
  VARIANT 5: note = Xaa can be Asp or Thr
GKXCXARDYYNWDFEH

SEQ ID NO: 81 - protein, 9 aa, synthetic construct
  REGION 1..9: note = Synthetic
  VARIANT 5: note = Xaa can be Ser or Tyr
RTSQXGSLA

SEQ ID NO: 82 - protein, 7 aa, synthetic construct
  REGION 1..7: note = Synthetic
SGSTRAA

SEQ ID NO: 83 - protein, 5 aa, synthetic construct
  REGION 1..5: note = Synthetic
QQYEF

SEQ ID NO: 84 - protein, 107 aa, Homo sapiens
EIVLTQSPATLSLSPGETAIISCRTSQYGSLAWYQQRPGQAPRLVIYSGSTRAAGIPDRFSGSRWGADYNLSISNLESGDFGVYYCQQYEFFGQGTKVQVDIKRTVA

SEQ ID NO: 85 - protein, 125 aa, Homo sapiens
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSS

(19) United States
(12) Patent Application Publication
(10) Pub. No.: US 2025/0066457 A1
(43) Pub. Date: Feb. 27, 2025

(54) ANTIBODIES TARGETING HIV ESCAPE MUTANTS

(71) Applicants: The Rockefeller University, New York, NY (US); California Institute of Technology, Pasadena, CA (US)

(72) Inventors: Ron Diskin, Pasadena, CA (US); Anthony P. West, Pasadena, CA (US); Michel C. Nussenzweig, New York, NY (US); Pamela J. Bjorkman, Altadena, CA (US)

(21) Appl. No.: 18/939,998

(22) Filed: Nov. 7, 2024

Related U.S. Application Data
(62) Division of application No. 17/500,171, filed on Oct. 13, 2021, now Pat. No. 12,173,052.
Application No. 17/500,171 is a continuation of application No. 15/835,319, filed on Dec. 7, 2017, now Pat. No. 11,149,081.
Application No. 15/835,319 is a continuation of application No. 13/924,469, filed on Jun. 21, 2013, now Pat. No. 9,879,068.
(60) Provisional application No. 61/662,594, filed on Jun. 21, 2012.

(51) Int. Cl. C07K 16/10; A61K 39/00

(52) U.S. Cl. CPC C07K 16/1063; C07K 16/1045; A61K 2039/505; A61K 2039/507; A61K 2039/545; C07K 2317/567; C07K 2317/76; C12N 2740/16111; C12N 2740/16122

(57) ABSTRACT

Embodiments of the present invention are directed to compositions and methods for anti-HIV (anti-CD4 binding site) broadly neutralizing antibodies having improved potency and breadth for neutralizing a range of HIV strains. Combinations of broadly neutralizing antibodies can also improve potency over a single antibody composition.

### CROSS-REFERENCE TO RELATED APPLICATION(S)

This application is a divisional of U.S. application Ser. No. 17/500,171, filed on Oct. 13, 2021, which is a continuation of U.S. application Ser. No. 15/835,319 filed on Dec. 7, 2017, now U.S. patent Ser. No. 11/149,081, which is a continuation of U.S. patent application Ser. No. 13/924,469, filed Jun. 21, 2013, now U.S. Pat. No. 9,879,068, which claims priority to and the benefit of U.S. Provisional Application Ser. No. 61/662,594, filed Jun. 21, 2012. The entire contents of these applications are incorporated herein by reference in their entirety.

### STATEMENT REGARDING FEDERALLY SPONSORED RESEARCH OR DEVELOPMENT

This invention was made with government support under Grant No. A1100148 and Grant No. A1100663 awarded by the National Institutes of Health. The government has certain rights in the invention.

### REFERENCE TO AN ELECTRONIC SEQUENCE LISTING

The contents of the electronic sequence listing (070413_20817SeqList.xml; Size: 88,441 bytes; and Date of Creation: Sep. 20, 2024) is herein incorporated by reference in its entirety.

### TECHNICAL FIELD

This application is directed to a gp120 anti-CD4 binding site (anti-CD4bs) antibody composition that has improved potency and breadth against the human immunodeficiency virus, (HIV) which causes acquired immunodeficiency syndrome (AIDS).

### BACKGROUND

Three decades after the emergence of HIV there is still no vaccine, and AIDS remains a threat to global public health. However, some HIV-infected individuals eventually develop broadly neutralizing antibodies (bNAbs), i.e., antibodies that neutralize a large panel of HIV viruses and that can delay viral rebound in HIV patients. Such antibodies are relevant to vaccine development, as evidenced by the prevention of infection observed after passive transfer to macaques.

The NIH45-46 antibody that was isolated in a screen using single cell cloning techniques (Scheid et al., 2009, J. Immunol Methods 343:65-67; Scheid et al., 2011, Science 333:1633-1637, the entire contents of both of which are herein incorporated by reference), is a more potent clonal variant of VRC01, a bNAb directed against the CD4 binding site (CD4bs) of gp120 (Wu et al., 2010, Science 329:856-861; and Zhou et al., 2010, Science 329:811-817, the entire contents of both of which are herein incorporated by reference). Enhancing the efficacy of bNAbs, and in particular, designing bNAbs that retain potency against escape mutants selected during exposure to bNAbs, would facilitate their use as therapeutics.

### SUMMARY

In some embodiments, a composition includes an isolated anti-CD4 binding site (anti-CD4bs) potentVRC01-like (PVL) antibody having a heavy chain and a light chain, the heavy chain including a first substitution at a position equivalent to Phe43 of a CD4 receptor protein, the heavy chain substitution being selected from the group consisting of glycine, histidine, arginine, glutamine, asparagine, lysine, glutamic acid, and aspartic acid; and a second substitution of tryptophan at position 47 of the heavy chain, selected from valine, isoleucine, and threonine; and the light chain including a substitution of tyrosine at position 28 of the light chain for serine.

In some embodiments a method of preventing or treating an HIV infection or an HIV-related disease includes administering a therapeutically effective amount of a composition, the composition including an isolated anti-CD4 binding site (anti-CD4bs) potentVRC01-like (PVL) antibody having a heavy chain and a light chain, the heavy chain including a first substitution at a position equivalent to Phe43 of a CD4 receptor protein, the heavy chain substitution being selected from the group consisting of glycine, histidine, arginine, glutamine, asparagine, lysine, glutamic acid, and aspartic acid; and a second substitution of tryptophan at position 47 of the heavy chain, selected from valine, isoleucine, and threonine; and the light chain including a substitution of tyrosine at position 28 of the light chain for serine.

In some embodiments, a method of preventing or treating an HIV infection or an HIV-related disease, the method comprising administering a therapeutically effective amount of at least two antibodies, the first antibody comprising the composition described above and the second antibody comprising 10-1074 antibody or PG9 antibody.

### BRIEF DESCRIPTION OF THE DRAWINGS

The patent or application file contains at least one drawing executed in color. Copies of this patent or patent application publication with color drawings will be provided by the Office upon request and payment of the necessary fee.

These and other features and advantages of the present invention will be better understood by reference to the following detailed description when considered in conjunction with the accompanying drawings.

FIG. 1 is a table listing NIH45-46m mutants, according to embodiments of the present invention.

FIG. 2 is a graph of coverage curves showing the cumulative frequency of IC50 values up to the concentration shown on the x-axis (plot of the percent of viral strains (y axis) from a panel of 118 HIV strains that were neutralized by NIH45-46, NIH45-46^{G54W}, 45-46m2, 46-46m7, and VRC01 at a given IC50 cut-off (x axis)); a vertical line at 0.1 μg/ml designates a theoretical desired potency for a therapeutic reagent, according to embodiments of the present invention.

FIG. 3 is a table showing IC₅₀ values (μg/ml) for NIH45-46, NIH45-46^{G54W}, 45-46m2 and 45-46m7 against 28 strains that are resistant to, or poorly neutralized by, NIH45-46. Strains marked in blue have an altered N/DNGG motif. IC₅₀s were derived from curves generated from data points obtained in duplicate or triplicate, according to embodiments of the present invention.

FIGS. 4A-4J are neutralization curves for NIH45-46^{G54W}, 45-46m2, and 45-46m7 against 10 viral clones (Clones 72, 113, 205, 01, 02, 03, 05, 06, 08, and 04 in FIGS. 4A-4J, respectively) from patient VC10042 that were isolated 19 years (first three panels) or 22 years (remaining panels) post infection, according to embodiments of the present invention.

FIG. 5A is a scatter plot comparing IC50 values (μg/ml) for VRC01, NIH45-46^{G54W}, 45-46m2 and 45-46m7 against viral clones from patient VC10042, according to embodiments of the present invention.

FIG. 5B is table of IC50 values (μg/ml) for NIH45-46^{G54W}, 45-46m2, and 45-46m7 against viral clones from patient VC10042, in which the reported IC50 values represent the average of two independent experiments, each with two replicates, according to embodiments of the present invention.

FIG. 6A is a schematic of the 45-46m2/gp120 structure with gp120 as a gray surface and 45-46m2 Fab in cyan (HC) and blue (LC) Cα traces, with ordered N-glycans shown in van der Waals representation, with the Asn276_{gp120}-linked N-glycan highlighted in shades of red, and Tyr28_{45-46m2(LC)} and TrP54_{45-46m2(HC)} are pointed with arrows, according to embodiments of the present invention.

FIG. 6B is a graphical representation of the buried surface areas between gp120 and the indicated antibodies, in which the buried surface area for NIH45-46^{G54W} was calculated by adding the contribution of Trp54 (derived from the structure of 45-46m2/gp120) to the buried surface area calculated from the NIH45-46/gp120 structure, according to embodiments of the present invention.

FIG. 6C is a close-up schematic comparison of the interactions of Trp54₄₅₋₄₆ₘ₂ (cyan sidechain) and Gly54_{NIH45-46} (magenta) with gp120 in the structures of 45-46m2/gp120 (gray) and NIH45-46/gp120 (magenta), showing a hydrogen bond (green dashed line) between the nitrogen atom of the Trp54₄₅₋₄₆ₘ₂ indole ring and the main chain carbonyl oxygen of Gly473_{gp120} creates a 4 Å shift (black arrow; Cα-Cα distance) of the gp120 main chain towards Trp54₄₅₋₄₆ₘ₂, and Ile371_{gp120} adopts a different rotamer to accommodate Trp54₄₅₋₄₆ₘ₂, according to embodiments of the present invention.

FIG. 6D is a schematic showing the electron density (green mesh; σ=2) for an N-linked glycan attached to Asn276_{gp120}, where a portion from the final model of the 45-46m2/gp120 complex is superimposed on a Fₒ-F_{c} electron density map calculated using the initial model prior to adding the glycan and after several rounds of simulated annealing refinement, according to embodiments of the present invention.

FIG. 6E is a close-up schematic of the Asn276_{gp120}-attached glycan and its interactions with the 45-46m2 LC (semi-transparent blue surface), in which the side chains of Tyr28₄₅₋₄₆ₘ₂, Trp65₄₅₋₄₆ₘ₂, Arg64₄₅₋₄₆ₘ₂ and Tyr89₄₅₋₄₆ₘ₂ are shown as sticks, according to embodiments of the present invention.

FIG. 7A shows the sensograms (orange curves) that were recorded for the interactions of injected 93TH057 gp120 produced in insect (Hi5) and mammalian (HEK293) cells over immobilized Fabs derived from the indicated antibodies in a 2-fold dilution series ranging from 500 nM-31 nM; where the kinetic constants (kₐ, kₐ) were derived from globally fitting the association and dissociation phases using a 1:1 binding model (black curves) and affinities were calculated as K_{D}=kₐ/kₐ; the residual plots (blue) within each sensogram describe the fit of the model to the data; and each binding experiment was conducted twice: once using gp120 produced in insect cells; and once using gp120 produced in mammalian cells, according to embodiments of the present invention.

FIG. 7B is a graph of the SPR measurements of 500 nM injected 93TH057 gp120 over the indicated immobilized Fabs, where each curve was normalized to its Rₘₐₓ, and the gray and white shaded areas designate the association and dissociation phases, respectively, according to embodiments of the present invention.

FIG. 8A is a schematic of steric constraints associated with the gp120 N/DNGG motif, in which an overview of loop D (green) and the V5 loop (magenta) of gp120 (gray) are interacting with the surface of the 45-46m2 HC (cyan) and LC (blue), and the CD4-binding loop of gp120 is shown in orange, according to embodiments of the present invention.

FIG. 8B is a schematic of the gp120 V5 loop region showing Gly458_{gp120} and Gly459_{gp120} with overlaid prediction of the consequences of aspartic acid substitutions at these positions (Asp458_{gp120} and Asp459_{gp120}; pink sticks), in which both aspartic acids could clash with Trp47_{45-46m2(HC)}, according to embodiments of the present invention.

FIG. 8C is a schematic of Asn279_{gp120} and Asn280gp120 (sticks and semi-transparent spheres) interactions with 45-46m2, in which a hydrogen bond (orange dashed line) between Asn279_{gp120} and the nitrogen atom of the Trp 102_{45-46m2(HC)} indole ring is shown, according to embodiments of the present invention.

FIG. 8D is a schematic showing possible steric clashes between a lysine or a tyrosine in gp120 positions 279 and 280 (pink) and Trp102_{45-46m2(HC)} and Trp47_{45-46m2(HC)}, according to embodiments of the present invention.

FIG. 8E is a stereo image showing modeled substitutions in the gp120 N/DNGG consensus sequence (Lys279_{gp120}, Tyr Asp458_{gp120}, and Asp459_{gp120}) at the interface with of gp120s with non-consensus substitutions, are shown together with Trp102_{45-46m2 HC} and Trp47_{45-46m2 HC}, according to embodiments of the present invention.

FIG. 9 is a table listing average IC₅₀ values (μg/ml) derived from in vitro neutralization assays for 45-4m antibodies against YU-2 mutants, in which three or more independent neutralization assays were performed for each mutant, according to embodiments of the present invention.

FIG. 10 is a table listing the IC₅₀ values (μg/ml) derived from in vitro neutralization assays for selected 45-4m antibodies against YU-2 mutant strains, in which five independent neutralization assays were performed for each mutant, according to embodiments of the present invention.

FIG. 11 shows neutralization curves for selected YU-2 mutant strains, as indicated, where the error bars represent standard deviation from the mean, according to embodiments of the present invention.

FIG. 12A shows a sequence alignment of YU-2, the two YU-2 Ala281_{gp120} mutants, and the three known HIV strains with a potential N-linked glycosylation site at Asn279_{gp120}, in which the glycosylation potential for Asn279_{gp120} was calculated for each strain using NetNGlyc 1.0 Server, according to embodiments of the present invention.

FIG. 12B shows replication profiles of YU-2 escape mutants from two independent experiments comparing the replication of various YU-2 escape mutants to YU-2 WT in PBMC cell culture, in which levels of virus in the supernatant were determined by measuring p24 levels at various time points after inoculation, and each value represents the average of two replicates each from two independent experiments, according to embodiments of the present invention.

FIG. 12C shows neutralization curves for 45-46m2 and 45-46m7 against YU-2A^{281T} and YU-2A281S, in which the curves for YU-2A^{281T} were derived using an extended concentration series, according to embodiments of the present invention.

FIG. 12D is a table of neutralization results of A281T-associated mutations affecting the Asn276_{gp120}-linked glycan, according to embodiments of the present invention.

FIG. 13A shows the results from HIV-1 therapy by a combination of two [45-46m2+45−46m7, labeled 45-46m2/m7] or three [45-46m2/m7+10-1074] bNAbs in HIV-1_{YU2}-infected humanized mice, in which the viral load is shown: the left panels show the viral load change from baseline (log₁₀ HIV-1 RNA copies/mL), and the right panels show the absolute viral load per mouse (RNA copies/mL), where each line represents a single mouse, and red arrows indicate start of antibody treatment; green lines, geometric average of untreated mice; red lines, geometric average of antibody treatment group indicated, the treatment groups were analyzed in parallel and reflect a single experiment comprising six control animals (untreated), eight mice treated with 45-46m2/m7, and six animals treated with the combination 45-46m2/m7+10-1074, according to embodiments of the present invention.

FIG. 13B is a graphical representation of the average viral load change (log₁₀ HIV-1 RNA copies/mL) from baseline at the indicated number of days from start of therapy (mean and standard error are shown), where the statistical test: Kruskal-Wallis test with Dunn's multiple comparison post-hoc test, asterisks (*p≤0.05; **p≤0.01) reflect statistically significant differences between the treatment groups indicated, according to embodiments of the present invention.

FIG. 13C shows two pie charts illustrating the distribution of amino acid changes in gp120 at sites targeted by NIH45-46^{G54W} (left; data from (Klein et al., 2012, as discussed and incorporated herein) versus the 45-46 (m2/m7) combination (right), in which the wedge sizes reflect the percent of gp120 sequences carrying the indicated resistance mutation at the time of viral rebound, and the center numbers refer to the number of mice (left) and the number of gp120 sequences (right) for each set of data, where the mutations listed within the A281T sector of the 45-46m2/m7 pie chart reflect compensatory mutations accompanying A281T, according to embodiments of the present invention.

FIGS. 14A-14B shows mutation analysis of gp120 sequences during antibody therapy, where FIG. 14A is the analysis for HIV-1_{YU2}-infected humanized mice treated with a combination of two [45-46m2+45-46m7, labeled 45-46m2/m7] bNAbs and FIG. 14B is the analysis for HIV-1_{YU2}-infected humanized mice treated with a combination of three [45-46m2/m7+10-1074] bNAbs and the sequences of gp120s from escape mutant viruses were determined; where individual gp120 nucleotide sequences are represented by horizontal gray bars with silent mutations indicated in green and replacement mutations in red; and shaded vertical lines indicate regions that allowed escape from NIH45-46^{G54W} (amino acid positions 280 and 459) and from 10-1074 (amino acid position 332); and all substitutions are relative to HIV-1_{YU2} (acc. number M93258) and numbered according to HXB2, according to embodiments of the present invention.

FIG. 15 is table of IC₅₀ values for NIH45-46^{G54W}, 45-46m2, and 45-46m7, 45-46m25 and 45-46m28 antibodies against the various HIV viral strains as indicated, according to embodiments of the present invention.

FIG. 16 is a table of IC₈₀ values for NIH45-46^{G54W}, 45-46m2, and 45-46m7, 45-46m25 and 45-46m28 antibodies tested against the indicated viral strains, according to embodiments of the present invention.

FIG. 17 is a table of the crystallographic data collection and refinement statistics for the 45-46m2/93TH057 crystal structure, according to embodiments of the present invention.

FIG. 18 is a table of IC₅₀ and IC₈₀ values for 45-46m2 antibody, 45-46m2/45-46m7 combined antibodies, 45-46m2/45-46m7/PG9 combined antibodies, and 45-46m2/45-46m7/10-1074 combined antibodies tested against the indicated viral strains, according to embodiments of the present invention.

FIGS. 19A-19B show mutation analysis of gp120 sequences during antibody therapy in which env sequences were cloned from mice treated with a combination of 45-46m2 and 45-46m7 as shown in FIG. 19A or 45-46m2, 45-46m7 and 10-1074, as shown in FIG. 19B, where dots indicate no change compared with the parental YU-2 sequence and mutations are indicated with a single-letter amino acid code; the three regions of Env that can potentially harbor escape mutations are shown; and the N/DNGG motif and position 332 (site of 10-1074-induced mutations) are highlighted in red, according to embodiments of the present invention.

FIG. 20A shows an alignment of the heavy chains of PVL antibodies, their less potent relatives, and their germ-line precursor (SEQ ID NOs. 2, 6, 8, 10, 12, 14, 16, 18, 20, 22, 24, 26, 28, 30, 32, 34, 36, 38, 40, 45, 46, and 50-62), according to embodiments of the present invention.

FIG. 20B shows an alignment of the light chains of PVL antibodies, their less potent relatives, and their germ-line precursors (SEQ ID NOs. 1, 5, 7, 9, 11, 13, 15, 17, 19, 21, 23, 25, 27, 29, 31, 33, 35, 37, 39, and 63-76), according to embodiments of the present invention.

### DETAILED DESCRIPTION

Structure-based design was previously used to create the NIH45-46^{G54W} antibody, a single amino acid change from the NIH45-46 antibody, which was previously the single most potent and broadly neutralizing anti-HIV-1 antibody described to date as disclosed in US Patent Publication 2012/0288502 to Diskin et al., 2010, Nature structural & molecular biology 17:608-613; Diskin et al., 2011, Science 334:1289-1293; Nakamura et al., 2013, AIDS 27:337-346; and Sather et al., 2012, J Virol 86:12676-12685 the entire contents of all of which are herein incorporated by reference). The NIH45-46^{G54W} antibody belongs to the PVL (Potent VRC01-Like) family of antibodies that target the CD4bs on the HIV-1 trimeric spike complex. The NIH45-46^{G54W} antibody was further substituted in the light chain with a tyrosine (Y) replacing the serine(S) at position 28. The resulting double substituted (i.e., “double mutant”) antibody is referred to as NIH45-46^{G54W} (HC) S28Y (LC) or as 45-46m2. This 45-46m2 antibody showed improved potency over NIH45-46^{G54W} as disclosed in U.S. patent application Ser. No. 13/714,398, the entire contents of which are herein incorporated by reference.

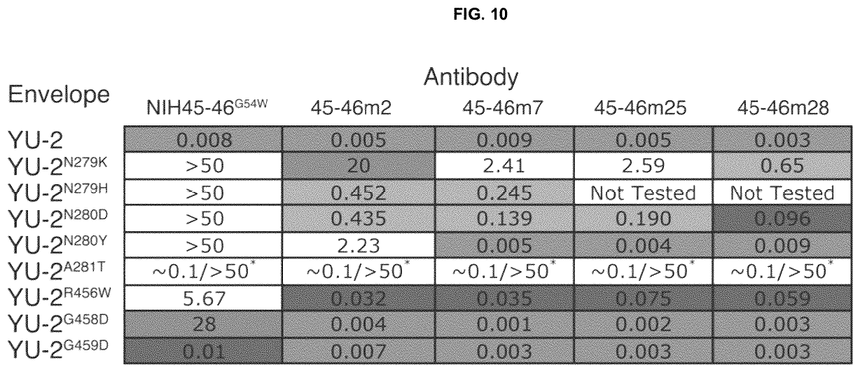

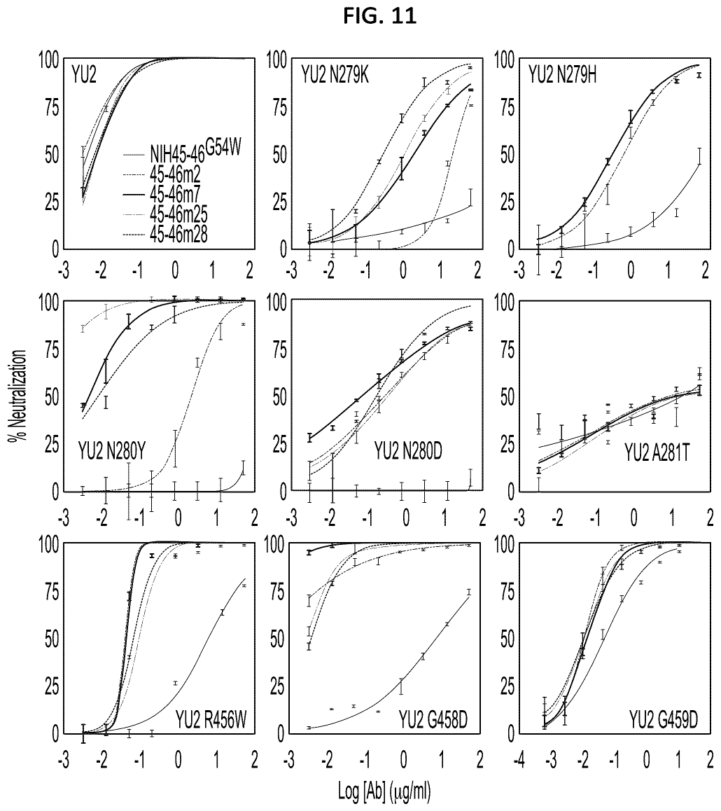

Nonetheless, a small group of HIV-1 clones are naturally resistant to neutralization by NIH45-46^{G54W} (Diskin et al., 2011, Science 334:1289-1293, the entire contents of which are herein incorporated by reference) and escape mutants emerge during exposure to NIH45-46^{G54W} (Klein et al., 2012 Nature 492:118-122, the entire contents of which are herein incorporated by reference). By replacing the highly conserved Trp47 residue (a germline residue) in the NIH45-46m2 (HC) antibody with different smaller amino acids, antibodies were identified that are capable of neutralizing strains YU-2N279K, YU-2N280D, and YU-2N280Y (FIGS. 10, 11). Specifically, the “triple” mutants, 45-46m7 (45-46m2+HC W47V), 45-46m25 (45-46m2+HC W471), and 45-46m28 (45-46m2+HC W47T), effectively neutralized all YU-2 mutants with the exception of YU-2A^{281T}, which included a newly introduced potential N-linked glycosylation site at Asn279_{gp120}.

In some embodiments, an antibody composition includes one of the triple mutants (45-46m7, 45-46m25, 45-46m28) combined with 45-46m2. In some embodiments, an antibody composition includes one of the triple mutants (45-46m7, 45-46m25, 45-46m28) combined with 45-46m2 and the PG9 antibody or the 10-1074 antibody, as described herein.

Abbreviations for amino acids are used throughout this disclosure and follow the standard nomenclature known in the art. For example, as would be understood by those of ordinary skill in the art, Alanine is Ala or A; Arginine is Arg or R; Asparagine is Asn or N; Aspartic Acid is Asp or D; Cysteine is Cys or C; Glutamic acid is Glu or E; Glutamine is Gln or Q; Glycine is Gly or G; Histidine is His or H; Isoleucine is Ile or I; Leucine is Leu or L; Lysine is Lys or K; Methionine is Met or M; Phenylalanine is Phe or F; Proline is Pro or P; Serine is Ser or S; Threonine is Thr or T; Tryptophan is Trp or W; Tyrosine is Tyr or Y; and Valine is Val or V.

Hydrophobic amino acids are well known in the art. Hydrophobic amino acids include alanine, isoleucine, leucine, methionine, phenylalanine, tryptophan, tyrosine, and valine. In some embodiments of the present invention, an anti-CD4bs PVL antibody has a hydrophobic amino acid substituted at a position equivalent to Phe43 of the CD4 receptor protein, wherein the hydrophobic amino acid is alanine, isoleucine, leucine, methionine, phenylalanine, tryptophan, tyrosine, or valine. In other embodiments, an anti-CD4bs PVL antibody has a hydrophobic amino acid substituted at the position equivalent to Phe43 of CD4 receptor protein, wherein the hydrophobic amino acid is tryptophan, phenylalanine, or tyrosine.

In addition to the hydrophobic acids, other amino acids that may be substituted at the Phe43-equivalent position of CD4 in the heavy chain of a PVL antibody, include glycine, histidine, arginine, glutamine, asparagine, glutamic acid, aspartic acid, lysine, and serine.

Throughout this disclosure and in embodiments of the present invention, the term “antibody” (Ab) as used herein includes monoclonal antibodies, polyclonal antibodies, multispecific antibodies (for example, bispecific antibodies and polyreactive antibodies), and antibody fragments. Thus, the term “antibody” and “isolated antibody” are used interchangeably herein to refer to an isolated antibody according to embodiments of the present invention. An antibody in any context within this specification is meant to include, but is not limited to, any specific binding member, immunoglobulin class and/or isotype (e.g., IgG1, IgG2, IgG3, IgG4, IgM, IgA, IgD, IgE and IgM); and biologically relevant fragment or specific binding member thereof, including but not limited to Fab, F (ab′) 2, Fv, and scFv (single chain or related entity). It is understood in the art that an antibody is a glycoprotein comprising at least two heavy (H) chains and two light (L) chains inter-connected by disulfide bonds, or an antigen binding portion thereof. A heavy chain is comprised of a heavy chain variable region (VH) and a heavy chain constant region (CH1, CH2 and CH3). A light chain is comprised of a light chain variable region (VL) and a light chain constant region (CL). The variable regions of both the heavy and light chains comprise framework regions (FWR) and complementarity determining regions (CDR). The four FWR regions are relatively conserved while CDR regions (CDR1, CDR2 and CDR3) represent hypervariable regions and are arranged from the NH2 terminus to the COOH terminus as follows: FWR1, CDR1, FWR2, CDR2, FWR3, CDR3, FWR4. The variable regions of the heavy and light chains contain a binding domain that interacts with an antigen while, depending on the isotype, the constant region(s) may mediate the binding of the immunoglobulin to host tissues or factors. CDR1, CDR2, and CDR3 of the light chain are referred to as CDRLI, CDRL2 and CDRL3, respectively. CDR1, CDR2, CDR3 of the heavy chain are referred to as CDRHI, CDRH2, and CDRH3, respectively.

Also included in the definition of “antibody” as used herein are chimeric antibodies, humanized antibodies, and recombinant antibodies, human antibodies generated from a transgenic non-human animal, as well as antibodies selected from libraries using enrichment technologies available to the artisan. The term “variable” refers to the fact that certain segments of the variable (V) domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta sheet configuration, connected by three hypervariable regions, which form loops connecting, and in some cases forming part of, the beta sheet structure. The hypervariable regions in each chain are held together in close proximity by the FRs and, with the hypervariable regions from the other chain, contribute to the formation of the antigen-binding site of antibodies. The term “hypervariable region” as used herein refers to the amino acid residues of an antibody that are responsible for antigen binding. The hypervariable region generally comprises amino acid residues from a “complementarity determining region” (“CDR”).

An antibody of the present invention may be a “humanized antibody”. A humanized antibody is considered to be a human antibody that has one or more amino acid residues introduced into it from a source that is non-human. These non-human amino acid residues often are referred to as “import” residues, which typically are taken from an “import” variable region. Humanization may be performed following known methods by substituting import hypervariable region sequences for the corresponding sequences of a human antibody. (See, for example, Jones et al., Nature, 321:522-525 20 (1986); Reichmann et al., Nature, 332:323-327 (1988); Verhocyen et al., Science, 239:1534-1536 (1988)) the entire contents of all of which are incorporated herein by reference). Accordingly, such “humanized” antibodies are chimeric antibodies in which less than a full intact human variable region has been substituted by the corresponding sequence from a non-human species.

An antibody of the present invention includes an “antibody fragment” which includes a portion of an intact antibody, such as the antigen binding or variable region of the intact antibody. Examples of antibody fragments include, but are not limited to, Fab, Fab′, F(ab′)2, and Fv fragments; diabodies; linear antibodies; single-chain antibody molecules; and multispecific antibodies formed from antibody fragments. (See, for example, U.S. Pat. No. 5,641,870, the entire content of which is incorporated herein by reference.)

Throughout this disclosure and in embodiments of the present invention, a “potent VRC01-like” (“PVL”) antibody of the present invention is an anti-CD4 binding site antibody that has the following conserved heavy chain (HC) and light chain (LC) residues: Arg71HC, Trp50_{HC}, Asn58_{HC}, Trp100B_{HC}, Glu96_{LC}, Trp67_{LC}/Phe67_{LC}, as well as exactly 5 amino acids in CDRL3 domain (using Kabat numbering). (The Kabat numbering system is described in Abhinandan, K. R. and Martin, A. C. R. (2008), “Analysis and improvements to Kabat and structurally correct numbering of antibody variable domains,” Molecular Immunology, 45:3832-3839, the entire contents of which are herein incorporated by reference.) A PVL antibody of the present invention is any antibody as defined herein, that has the listed PVL features irrespective of the synthesis or derivation of the antibody, irrespective of the other unrestricted domains of the antibody, and irrespective of whether or not other domains of the antibody are present, so long as the antibody has the signature residues and features.

Throughout the disclosure and in embodiments of the present invention, the terms “Phe43-equivalent position” and “Phe43_{CD4} equivalent position” are used interchangeably and refer to an amino acid position within the heavy chain of a PVL antibody that replicates or mimics the binding pocket and interface contributed by Phe43 of the host CD4 receptor when the CD4 receptor protein is complexed with the HIV viral spike protein gp120. As known in the art, assigned amino acid positions of an antibody do not necessarily correspond to the amino acid residue as numbered from the amino-terminus. Following the Kabat antibody residue/position numbering system, the amino acid residue number may be the same as the amino acid position, but is not necessarily so. (Sec, Abhinandan, K. R. and Martin, A. C. R. (2008) Molecular Immunology, 45:3832-3839.) The structure of the antibody peptide determines the position number. The information for determining position number using the Kabat system for each amino acid in a given sequence can be determined using the information found in Abhinandan and Martin, 2008. Using this position numbering system, the Phe43-equivalent position in a PVL antibody heavy chain sequence can be determined, and substituted with a hydrophobic amino acid to create a similar binding pocket as conferred by Phe43 in CD4. Methods for this mutagenesis are well known in the art.

Subsequent heavy chain sequences can be analyzed using the Kabat numbering system to determine the equivalent position to this position 54. Alternatively, the Phe43_{CD4}-equivalent position can also be determined by structural analysis such as x-ray crystallography. Any means of determining the Phe43_{CD4}-equivalent position may be used so long as the Kabat system is followed as applicable.

For example, the Phe43-equivalent position in NIH45-46 is position 54 as determined by x-ray crystallography and shown herein. The native NIH45-46 heavy chain sequence (SEQ ID NO: 6) contains a glycine at position 54 (Gly54). The native 3BNC60 heavy chain sequence (SEQ ID NO: 8) contains a threonine at position 54 (Thr54). As such, these PVL antibodies substituted with a hydrophobic amino acid, glycine, histidine, arginine, glutamine, or asparagine at these Phe-43 equivalent positions mimic the desired contact interface between the CD4 receptor protein and the CD4 binding site of gp120 (see, e.g., Example 2).

In some embodiments of the present invention, position 54 (Kabat numbering) of the heavy chain of a PVL antibody has a substituted hydrophobic amino acid. Position 54 is determined by analyzing a heavy chain amino acid sequence of a PVL antibody using the Kabat numbering system.

In some embodiments of the present invention, a hydrophobic amino acid is substituted for the “native” amino acid present at the Phe43_{CD4}-equivalent position on the heavy chain of a PVL antibody, where a PVL antibody is an antibody as defined herein having the PVL signature features as described herein, and “native” refers to the amino acid that is present in the PVL antibody prior to substitution. The native amino acid in the heavy chain may also be hydrophobic, and may be substituted with another hydrophobic amino acid, or with glycine, histidine, arginine, glutamine, asparagine, lysine, glutamic acid, or aspartic acid.

In some embodiments of the present invention, non-limiting examples of PVL antibodies include VRC01, VRC02, NIH-45-46, 3BNC60, 3BNC117, 3BNC62, 3BNC95, 3BNC176, 12A21, VRC-PG04, VRC-CH30, VRC-CH31, VRC-CH32, VRC-CH33, VRC-CH34, VRC03 heavy chain (HC) with VRC01 light chain (LC), gVRC-H5 (d74)/VRC-PG04LC, and gVRC-H12(d74)/VRC-PG04LC. VRC03. VRC01 heavy chain (HC) with VRC03 light chain (LC). 3BNC55. 3BNC91. 3BNC104. 3BNC89, 12A21. and VRC-PG04b as listed below in Table 1.

TABLE 1
Examples of PVL Antibodies

| | Light Chain | Heavy Chain |
|---|---|---|
| Antibody Name | SEQ ID NO: | SEQ ID NO: |
| VRC01 | 1 | 2 |
| VRC02 | 3 | 4 |
| NIH-45-46 | 5 | 6 |
| 3BNC60 | 7 | 8 |
| 3BNC117 | 9 | 10 |
| 3BNC62 | 11 | 12 |
| 3BNC95 | 13 | 14 |
| 3BNC176 | 15 | 16 |
| 12A12 | 17 | 18 |
| VRC-PG04 | 19 | 20 |
| VRC-CH30 | 21 | 22 |
| VRC-CH31 | 23 | 24 |
| VRC-CH32 | 25 | 26 |
| VRC-CH33 | 27 | 28 |
| VRC-CH34 | 29 | 30 |
| VRC03 | 31 | 32 |
| 3BNC55 | 33 | 34 |
| 3BNC91 | 35 | 36 |
| 3BNC104 | 37 | 38 |
| 3BNC89 | 39 | 40 |
| 12A21 | 41 | 42 |
| VRC-PG04b | 43 | 44 |
| VRC03HC- VRC01LC | 1 | 32 |
| VRC01HC/VRC03LC | 31 | 2 |
| gVRC-H5(d74)/ | 19 | 45 |
| VRC-PG04LC | | |
| gVRC0H12(D74)/ | 19 | 46 |
| VRC-PG04LC | | |

In some embodiments of the present invention, a PVL antibody has a heavy chain selected from one of the heavy chains listed above in Table 1 (SEQ ID NOs 2, 4, 6, 8, 10, 12, 14, 16, 18, 20, 22, 24, 26, 28, 30, 32, 34, 36, 38, 40, 42, 44, 45, and 46). Any PVL heavy chain may be matched with a PVL light chain so long as the signature PVL residue features are maintained. In some embodiments, any one of the PVL heavy chains of Table 1 is expressed with any one of the PVL light chains of SEQ ID NOs 1, 3, 5, 7, 9, 11, 13, 15, 17, 19, 21, 23, 25, 27, 29, 31, 33, 35, 37, 39, 41, and 43. In other embodiments, any PVL antibody heavy chain can be combined with any PVL antibody light chain.

In embodiments of the present invention, the terms “nucleic acid” and “polynucleotide” are used interchangeably herein to refer to single-stranded or double-stranded RNA, DNA, or mixed polymers. Polynucleotides can include genomic sequences, extra-genomic and plasmid sequences, and smaller engineered gene segments that express, or can be adapted to express polypeptides.

An “isolated nucleic acid” is a nucleic acid that is substantially separated from other genome DNA sequences as well as proteins or complexes such as ribosomes and polymerases, which naturally accompany a native sequence.

In some embodiments of the present invention, nucleic acid molecules encode part or all of the light and heavy chains of the described inventive antibodies, and fragments thereof. Due to redundancy of the genetic code, variants of these sequences will exist that encode the same amino acid sequences.

The present invention also includes isolated nucleic acid molecules encoding the polypeptides of the heavy and the light chain of the PVL antibodies listed in Table 1. In some embodiments, an isolated nucleic acid molecule encodes for any of the PVL heavy chain and light chain polypeptides including those of SEQ ID NOs 2, 4, 6, 8, 10, 12, 14, 16, 18, 20, 22, 24, 26, 28, 30, 32, 34, 36, 38, 40, 42, 44, 45, and 46, and SEQ ID NOs 1, 3, 5, 7, 9, 11, 13, 15, 17, 19, 21, 23, 25, 27, 29, 31, 33, 35, 37, 39, 41, and 43, respectively, in which the Phe43CD4-equivalent amino acid (i.e., the target amino acid) of the heavy chain is substituted with a hydrophobic amino acid.

Embodiments of the present invention also include vectors and host cells including a nucleic acid encoding a PVL antibody of the present invention, as well as recombinant techniques for the production of polypeptide of the invention. Vectors of the invention include those capable of replication in any type of cell or organism, including, for example, plasmids, phage, cosmids, and mini chromosomes. In some embodiments, vectors comprising a polynucleotide 5 of the described invention are vectors suitable for propagation or replication of the polynucleotide, or vectors suitable for expressing a polypeptide of the described invention. Such vectors are known in the art and commercially available.

In embodiments of the present invention, “vector” includes shuttle and expression vectors. Typically, the plasmid construct will include an origin of replication (for example, the ColE1 origin of replication) and a selectable marker (for example, ampicillin or tetracycline resistance), for replication and selection, respectively, of the plasmids in bacteria. An “expression vector” refers to a vector that contains the necessary control sequences or regulatory elements for expression of the antibodies including antibody fragment of the invention, in bacterial or eukaryotic cells.

In some embodiments of the present invention, in order to express a polypeptide of the invention, the nucleotide sequences encoding the polypeptide, or functional equivalents, may be inserted into an appropriate expression vector, i.e., a vector that contains the necessary elements for the transcription and translation of the inserted coding sequence. Methods well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J., et al. (2001) Molecular Cloning, A Laboratory Manual, Cold Spring Harbor Press, Plainview, N.Y., the entire contents of which are incorporated herein by reference.

As used herein, the term “cell” can be any cell, including, but not limited to, eukaryotic cells, such as, but not limited to, mammalian cells or human cells.

In some embodiments of the present invention, the antibodies disclosed herein are produced recombinantly using vectors and methods available in the art. (See, e.g. Sambrook et al., 2001, supra). Human antibodies also can be generated by in vitro activated B cells. (See, for example, U.S. Pat. Nos. 5,567,610 and 5,229,275, the entire contents of both of which are herein incorporated by reference.) Reagents, cloning vectors, and kits for genetic manipulation are available from commercial vendors such as BioRad, Stratagene, Invitrogen, ClonTech and Sigma-Aldrich Co.

In some embodiments of the present invention, human antibodies are produced in transgenic animals (for example, mice) that are capable of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germline mutant mice results in the production of human antibodies upon antigen challenge. See, for example, Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669; 5,545,807; and WO 97/17852, the entire contents of all of which are herein incorporated by reference. Such animals can be genetically engineered to produce human antibodies comprising a polypeptide of a PVL antibody according to embodiments of the present invention.

In some embodiments of the present invention, a method includes the preparation and administration of an HIV antibody composition (e.g., a PVL antibody having a hydrophobic amino acid substituted at the Phe43_{CD4}-equivalent position of the PVL heavy chain) that is suitable for administration to a human or non-human primate patient having an HIV infection, or at risk of HIV infection, in an amount and according to a schedule sufficient to induce a protective immune response against HIV, or reduction of the HIV virus, in a human.

In some embodiments of the present invention, a vaccine includes at least one antibody as disclosed herein and a pharmaceutically acceptable carrier. In some embodiments of the present invention, the vaccine is a vaccine including at least one PVL antibody as described herein and a pharmaceutically acceptable carrier. The vaccine can include a plurality of the antibodies having the characteristics described herein in any combination and can further include HIV neutralizing antibodies such as a PVL antibody having the Phe43_{CD4}-equivalent residue on the heavy chain substituted with a hydrophobic amino acid.

In some embodiments of the present invention, carriers as used herein include pharmaceutically acceptable carriers, excipients or stabilizers that are nontoxic to a cell or mammal being exposed thereto at the dosages and concentrations employed. Often the physiologically acceptable carrier is an aqueous pH buffered solution. Examples of physiologically acceptable carriers include, but are not limited to, buffers such as phosphate, citrate, and other organic acids; antioxidants including, but not limited to, ascorbic acid; low molecular weight (less than about 10 residues) polypeptides; proteins, such as, but not limited to, serum albumin, gelatin, or immunoglobulins; hydrophilic polymers such as, but not limited to polyvinylpyrrolidone; amino acids such as, but not limited to glycine, glutamine, asparagine, arginine or lysine; monosaccharides, disaccharides, and other carbohydrates including, but not limited to glucose, mannose, or dextrins; chelating agents such as, but not limited to EDTA (ethylenediamineteteraacetic acid); sugar alcohols such as, but not limited to mannitol or sorbitol; salt-forming counterions such as, but not limited to sodium; and/or nonionic surfactants such as, but not limited to TWEEN® (polysorbate); polyethylene glycol (PEG), and PLURONICS® (poloxamers).

In some embodiments of the present invention, the compositions may include a single antibody or a combination of antibodies, which can be the same or different, in order to prophylactically or therapeutically treat the progression of various subtypes of HIV infection after vaccination. Such combinations can be selected according to the desired immunity. When an antibody is administered to an animal or a human, it can be combined with one or more pharmaceutically acceptable carriers, excipients or adjuvants as are known to one of ordinary skill in the art. The composition can further include broadly neutralizing antibodies known in the art, including, for example, a PVL antibody having the Phe43_{CD4}-equivalent residue substituted with a hydrophobic amino acid or glycine, histidine, arginine, glutamine, asparagine, lysine, glutamic acid, or aspartic acid, and the serine at position 28 of the light chain substituted with tyrosine (S28Y LC).

In some embodiments of the present invention, an antibody-based pharmaceutical composition includes a therapeutically effective amount of an isolated HIV antibody which provides a prophylactic or therapeutic treatment choice to reduce infection of the HIV virus. The antibody-based pharmaceutical composition according to embodiments of the present invention may be formulated by any number of strategies known in the art (e.g., see McGoff and Scher, 2000, Solution Formulation of Proteins/Peptides: In McNally, E. J., ed. Protein Formulation and Delivery. New York, N. Y.: Marcel Dekker; pp. 139-158; Akers and Defilippis, 2000, Peptides and Proteins as Parenteral Solutions. In: Pharmaceutical Formulation Development of Peptides and Proteins. Philadelphia, Pa.: Taylor and Francis; pp. 145-177; Akers, et al., 2002, Pharm. Biotechnol. 14:47-127, the entire contents of all of which are incorporated herein by reference).

In some embodiments of the present invention, a method for treating a mammal infected with a virus infection, such as, for example, HIV, includes administering to said mammal a pharmaceutical composition including an HIV antibody composition according to an embodiment disclosed herein. According to some embodiments, the method for treating a mammal infected with HIV includes administering to said mammal a pharmaceutical composition that includes an antibody according to an embodiment disclosed herein, or a fragment thereof. The compositions of embodiments of the present invention may include more than one antibody having the characteristics disclosed herein (for example, a plurality or pool of PVL antibodies, each antibody having the Phe43_{CD4}-equivalent residue substituted with a hydrophobic amino acid).

In some embodiments of the present invention, in vivo treatment of human and non-human patients includes administering or providing a pharmaceutical formulation including an HIV antibody according to embodiments of the present invention. When used for in vivo therapy, the antibodies of the invention are administered to the patient in therapeutically effective amounts (i.e., amounts that eliminate or reduce the patient's viral burden). The antibodies are administered to a human patient, in accord with known methods, such as intravenous administration, for example, as a bolus or by continuous infusion over a period of time, by intramuscular, intraperitoneal, intracerobrospinal, subcutaneous, intra-articular, intrasynovial, intrathecal, oral, topical, or inhalation routes. The antibodies can be administered parenterally, when possible, at the target cell site, or intravenously. In some embodiments, a PVL antibody composition according to embodiments as described herein is administered by intravenous or subcutaneous administration.

In some embodiments of the present invention, a therapeutically effective amount of an antibody is administered to a patient. In some embodiments, the amount of antibody administered is in the range of about 0.1 mg/kg to about 50 mg/kg of patient body weight. Depending on the type and severity of the infection, about 0.1 mg/kg to about 50 mg/kg body weight (for example, about 0.1-15 mg/kg/dose) of antibody is an initial candidate dosage for administration to the patient, whether, for example, by one or more separate administrations, or by continuous infusion. The progress of this therapy is readily monitored by conventional methods and assays and based on criteria known to the physician or other persons of skill in the art. The above parameters for assessing successful treatment and improvement in the disease are readily measurable by routine procedures familiar to a physician.

In some embodiments of the present invention, passive immunization using a PVL antibody according to embodiments as disclosed herein, is used as an effective and safe strategy for the prevention and treatment of HIV disease. (See, for example, Keller et al., Clin. Microbiol. Rev. 13:602-14 (2000); Casadevall, Nat. Biotechnol. 20:114 (2002); Shibata et al., Nat. Med. 5:204-10 (1999); and Igarashi et al., Nat. Med. 5:211-16 (1999), the entire contents of all of which are herein incorporated by reference).

The following Examples are presented for illustrative purposes only, and do not limit the scope or content of the present application.

### EXAMPLES

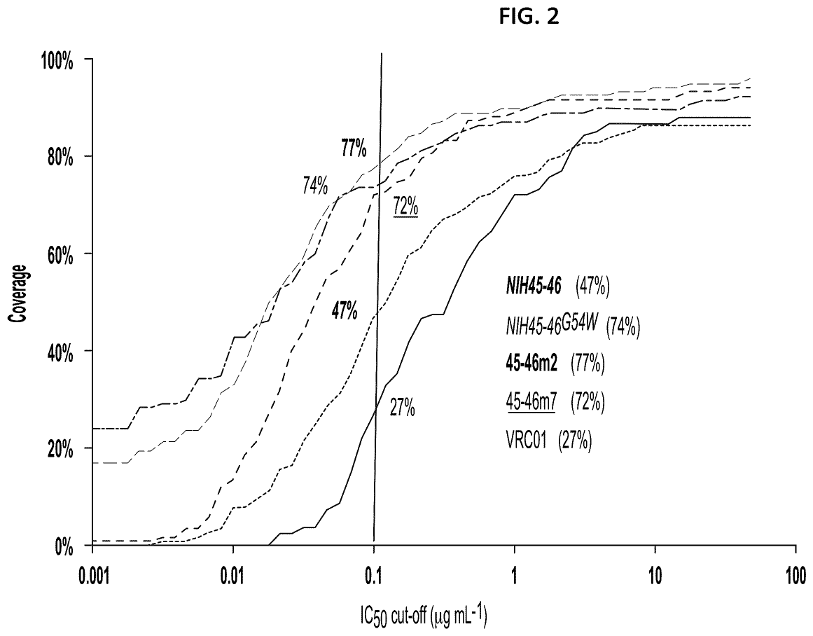

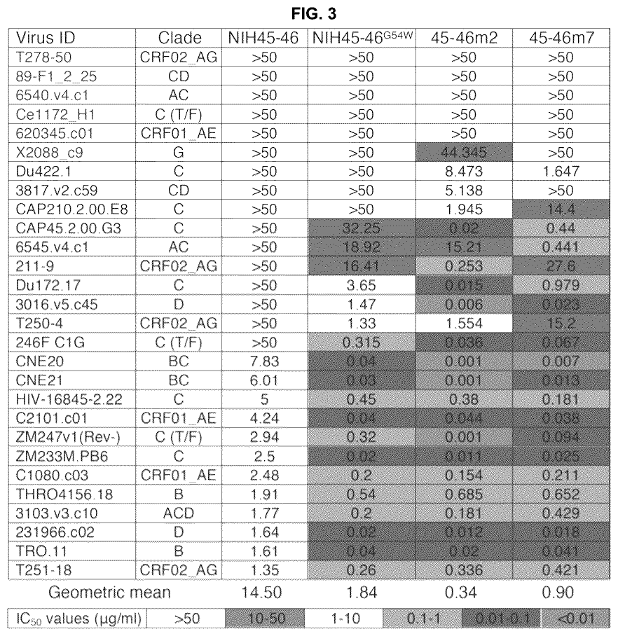

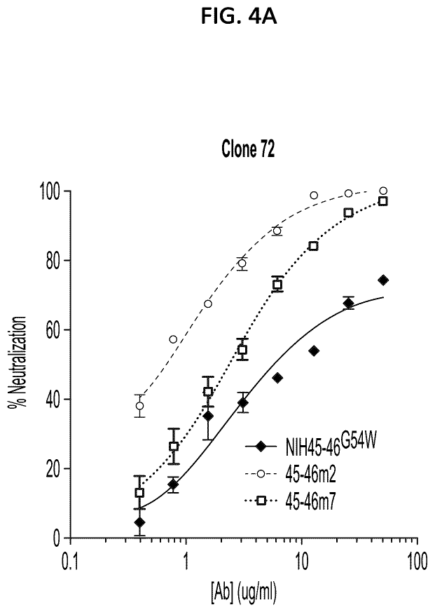

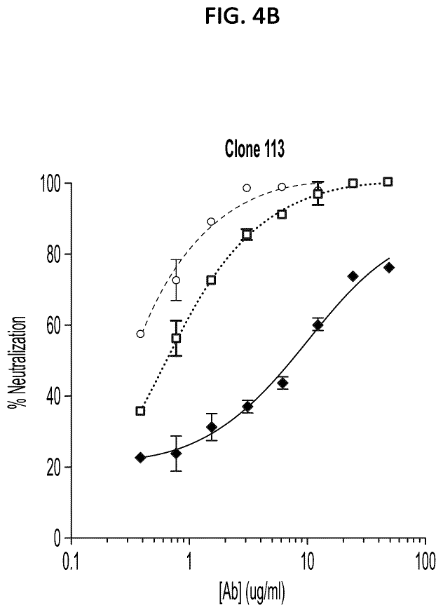

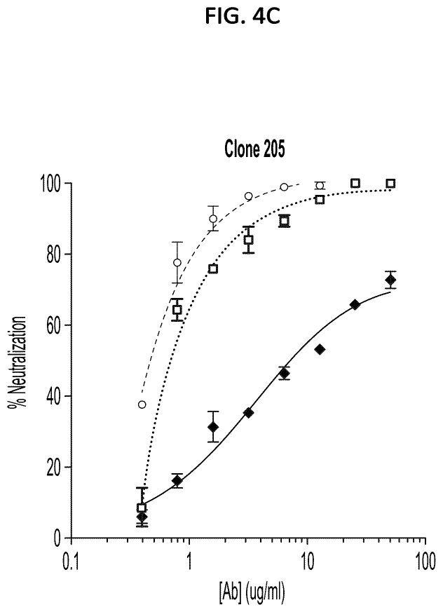

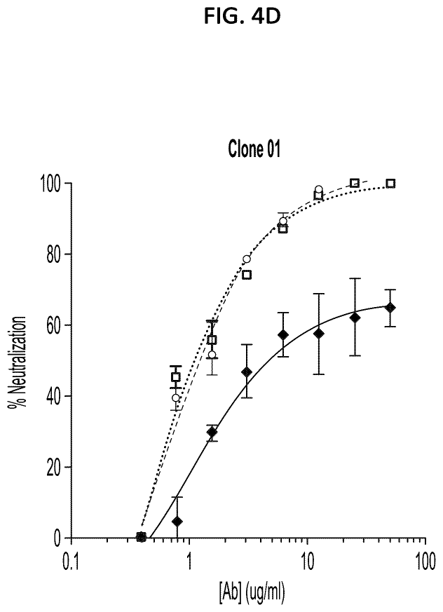

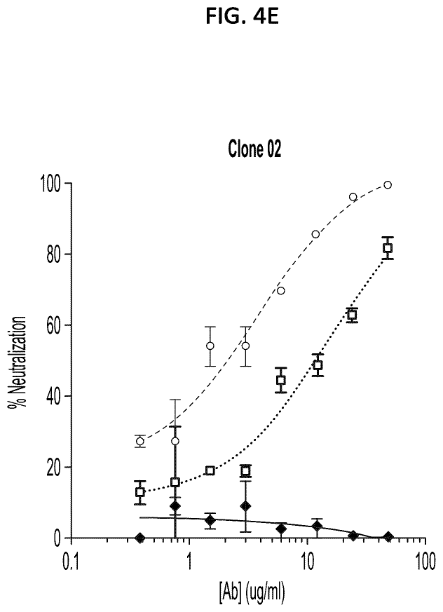

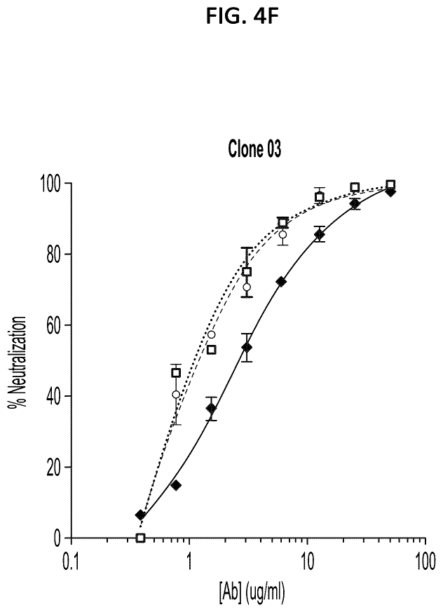

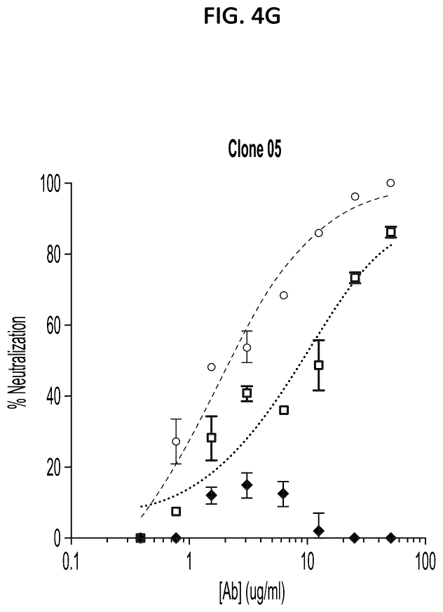

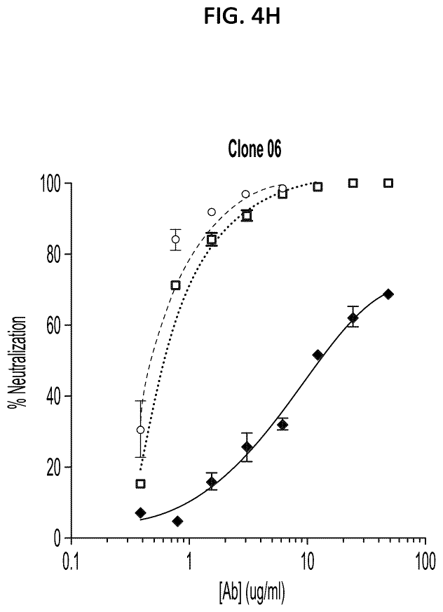

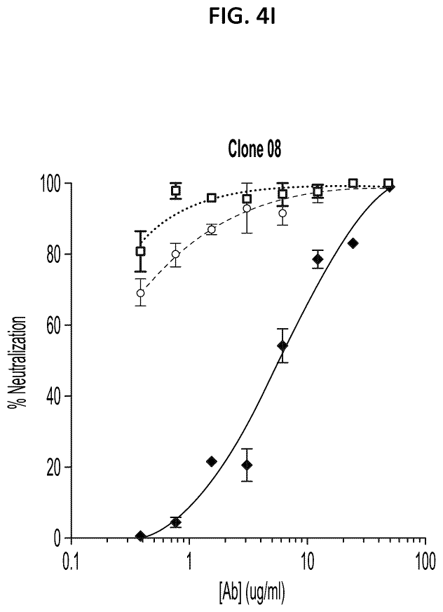

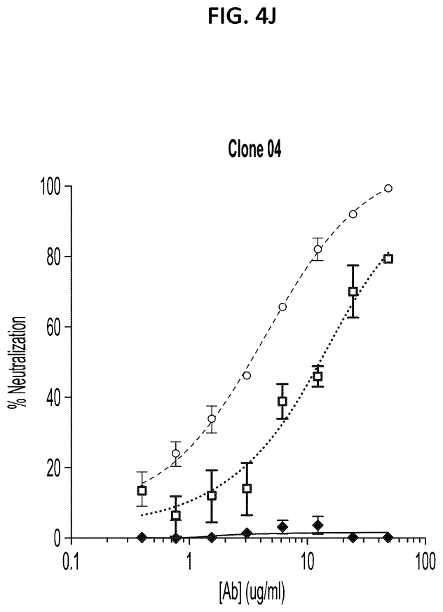

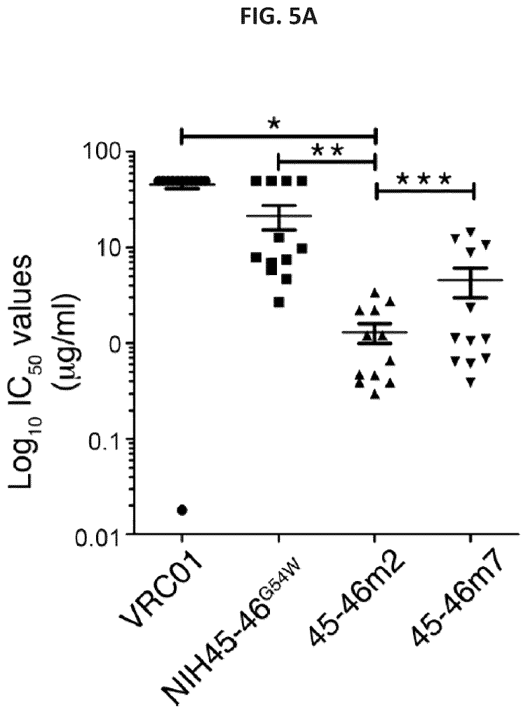

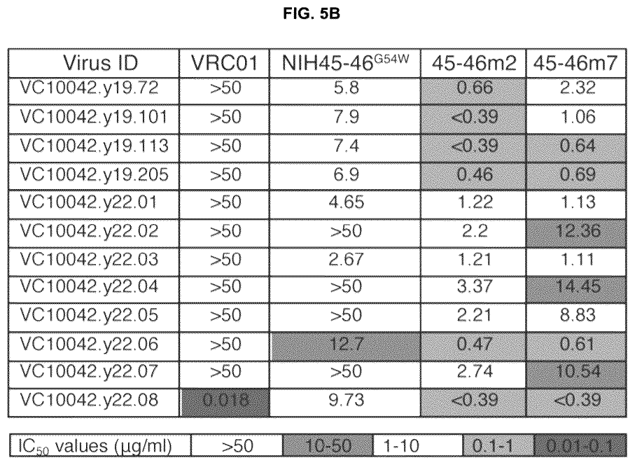

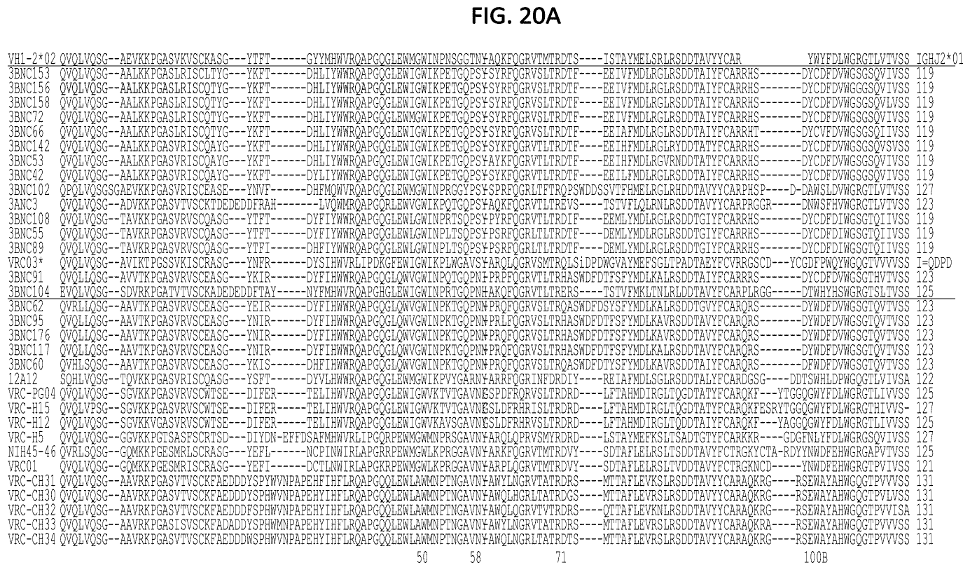

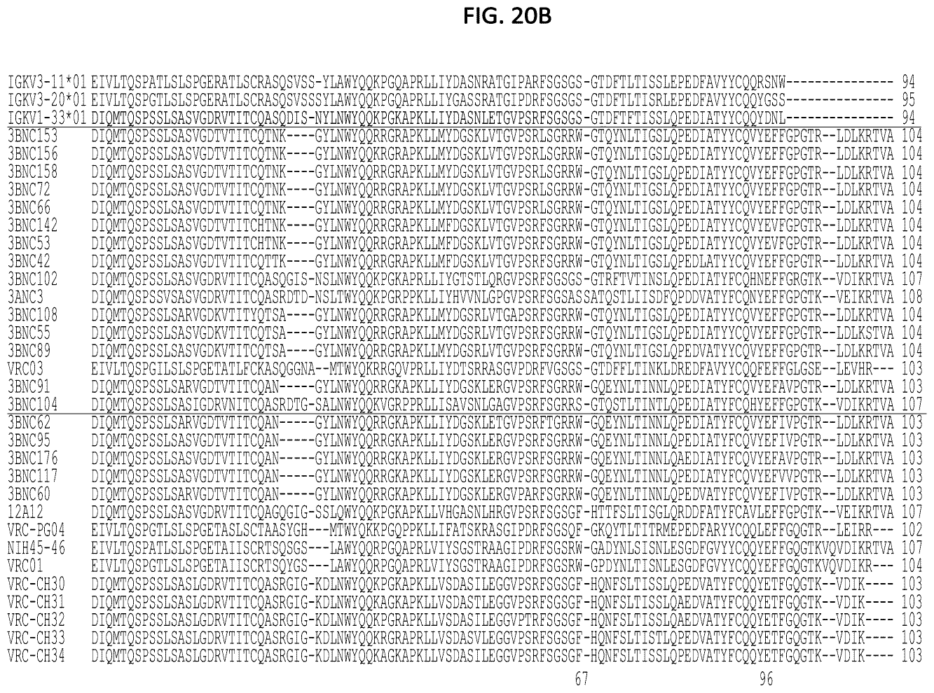

Reference is made to Diskin et al. 2013, JEM, 210:1235-1249; Diskin et al., 2011, Science, 334:12989-1293; and West et al., 2012, PNAS, (doi: 10.1073/pnas. 1208984109), the entire contents of all of which are incorporated herein by reference. FIGS. 20A and 20B show the heavy chain (SEQ ID NOs. 2, 6, 8, 10, 12, 14, 16, 18, 20, 22, 24, 26, 28, 30, 32, 34, 36, 38, 40, 45, 46, and 50-62) and light chain (SEQ ID NOs. 1, 5, 7, 9, 11, 13, 15, 17, 19, 21, 23, 25, 27, 29, 31, 33, 35, 37, 39, and 63-76) amino acid sequence alignments of several related variant groups of PVL antibodies as presented in FIG. 2 of West et al. with CDRs defined using the Chothia definition of the Abysis database.

### Example 1. Targeting Emerging Escape Mutants

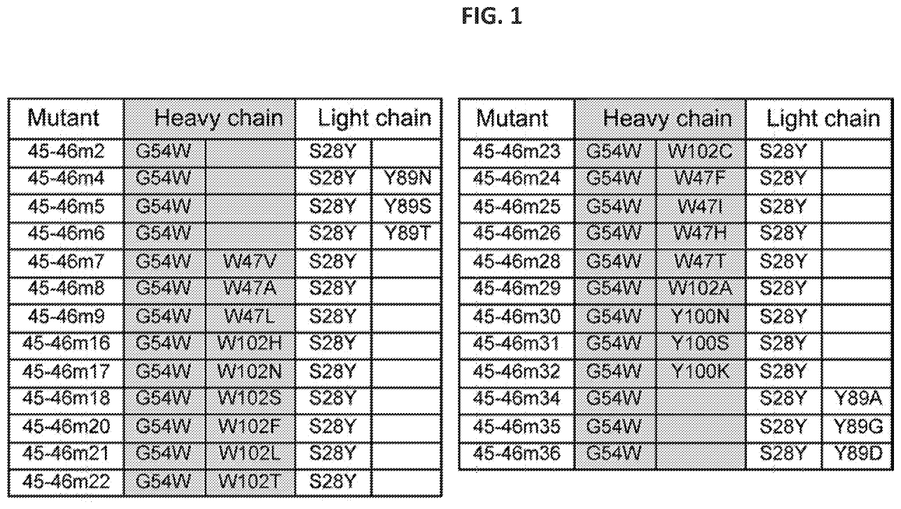

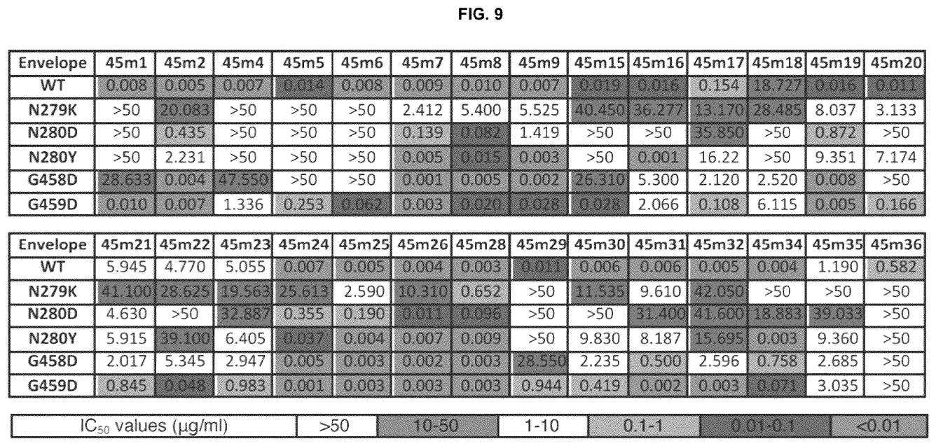

To further improve the potency of 45-46m2 against escape mutants in general and against non-consensus N/DNGG motifs in particular, 24 antibody mutants were designed to reduce steric clashes between 45-46m2 and substituted residues in the gp120 N/DNGG motif (FIG. 1). The neutralization potencies of the new mutant antibodies were evaluated against the panel of YU-2 mutants (FIG. 9-10). Modifying critical somatically-mutated residues in PVL antibodies (Trp 102_{45-46m2(HC)}, Tyr 100_{45-46m2(HC)} and Tyr89_{45-46m2(LC)}) (West et al., 2012, supra) to create mutants 45-46m4, m5, m6, m16, m17, m18, m20, m21, m22, m23, m29, m30, m31, m32, m34, m35 and m36 did not improve the neutralization profiles of the antibodies (FIG. 9). However, replacing the highly conserved Trp47_{45-46m2(HC)} (a germline residue) with different smaller amino acids resulted in antibodies capable of neutralizing YU-2N279K, YU-2N280D, and YU-2N280Y (FIGS. 10-11). These mutants, 45-46m7, 45-46m25, and 45-46m28 (45-46m2+HC mutations W47V, W47I, and W47T, respectively), effectively neutralized all YU-2 mutants with the exception of YU-2A28IT, which included a newly introduced potential N-linked glycosylation site at Asn279_{gp120.}

### Example 2. Fitness Cost Associated with a Glycan at Asn279_{gp120}

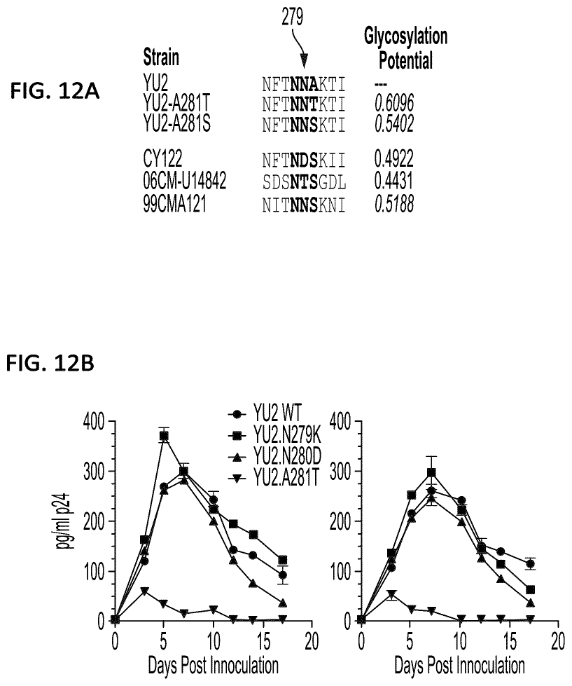

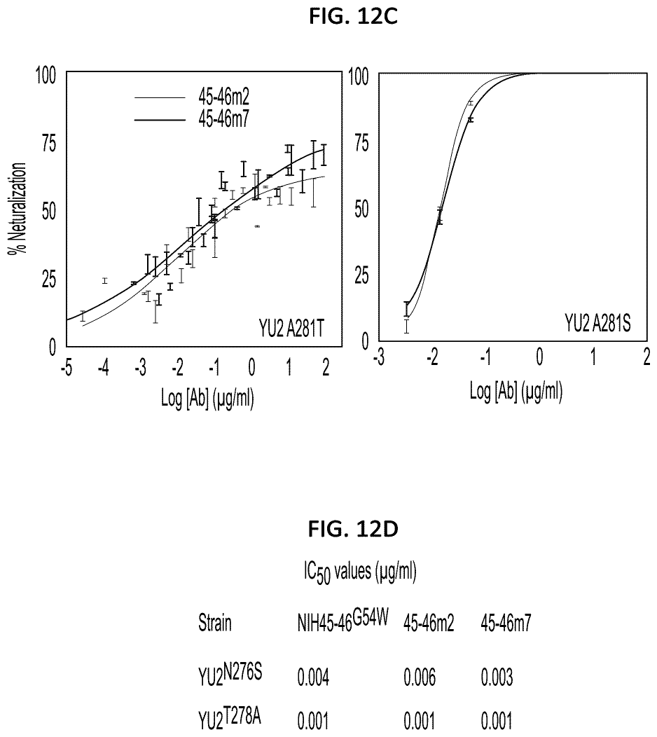

To explore the effects of the A281T_{gp120} mutation that abrogated neutralization of the 45-46m antibodies, a previously-described in vitro assay (Sather et al., 2012, J Virol 86:12676-12685, the entire contents of which are herein incorporated by reference) was used to compare the relative fitness of YU-2 mutants that either included (YU-2_{A281T}) or did not include (YU-2 WT, YU-2^{N279K}, and YU-2^{N280D}) a potential N-linked glycosylation site (Asn279_{gp120}-X-Ser/Thr281_{gp120}) (FIG. 12A). Although infectious, YU-2A28IT exhibited a disrupted replication profile relative to the other viruses (FIG. 12B), suggesting a fitness cost associated with an Asn279_{gp120}-attached glycan. Consistent with this suggestion, only three strains in the Los Alamos Data Base carry a potential N-linked glycosylation site at Asn279_{gp120}, all with low predicted glycosylation potentials (FIG. 12A). In addition, unlike the curves for other YU-2 variants, the in vitro neutralization curves for YU-2A281T saturated at about 50%, suggesting the existence of heterogeneous viral populations resulting from incomplete incorporation of N-linked glycan at Asn279_{gp120} (FIG. 11; FIG. 12C).

Available structural information about CD4 binding to gp120 can be used to rationalize why the viral replication profiles of the YU-2A^{281T} mutant were retarded compared to YU-2 WT (FIG. 12B). The N-linked glycan attached to Asn279_{gp120} that was introduced by the A281T_{gp120} substitution is predicted to exert a fitness cost for HIV-1 by partially blocking the CD4 binding site. Indeed if there was not a fitness cost associated with having a glycan at residue 279, one would not expect the highly correlated amino acid distribution at sites 279 and 281 observed in the Los Alamos database. Specifically, residue 279_{gp120} is usually Asn (51%) or Asp (46%), and this preference is not clade-specific. Residue 281_{gp120} is Thr in about 11% (n=314) of the sequences. However, Thr (T) occurs in only one strain (CH080183_e_p1) that includes Asn at 279_{gp120}, a distribution that has less than a 1 in 10¹⁰⁰ chance of occurring randomly (Fisher Exact test). Furthermore the middle residue of the potential N-linked glycosylation sequence in this strain is proline, which would prevent attachment of an N-glycan to Asn279_{gp120}. Additionally, of the 39 strains with Ser281_{gp120}, only three have Asn279_{gp120} (CY122, 99CMA121, U14842). An analysis of the glycosylation potential of these three strains (FIG. 12A) using the NetNGlyc 1.0 Server indicates no glycosylation potential (strains CY122 and U14842) or a very low potential (strain 99CMA121) for Asn279_{gp120}. The YU-2^{A281S} mutant has a higher glycosylation potential at Asn279_{gp120} compared with the 99CMA121 strain (0.5402 vs. 0.5188; FIG. 12A) but unlike YU-2A28IT, the YU-2A28IS mutant was neutralized well (FIG. 12C), suggesting that an N-glycan was not incorporated at Asn279_{gp120} despite the N-X-S motif.

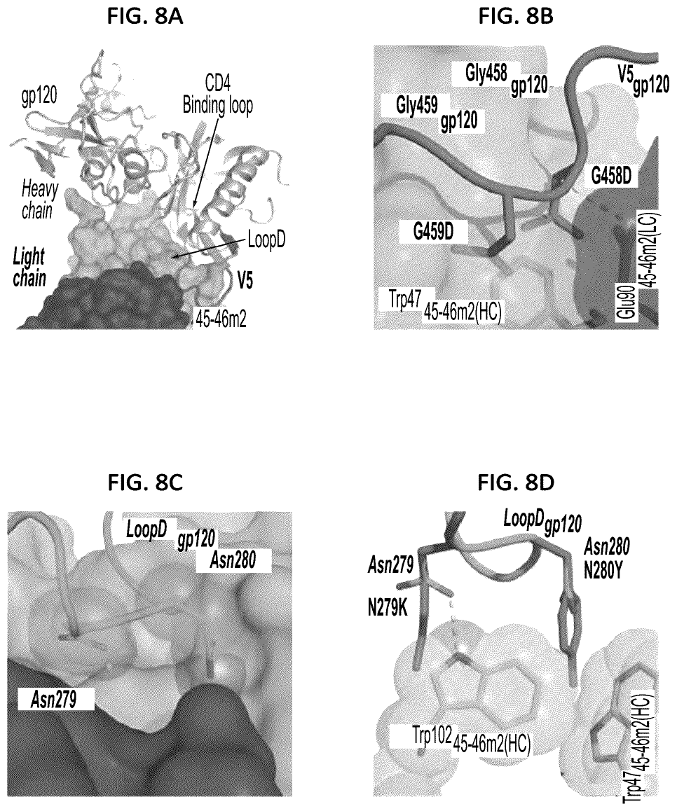

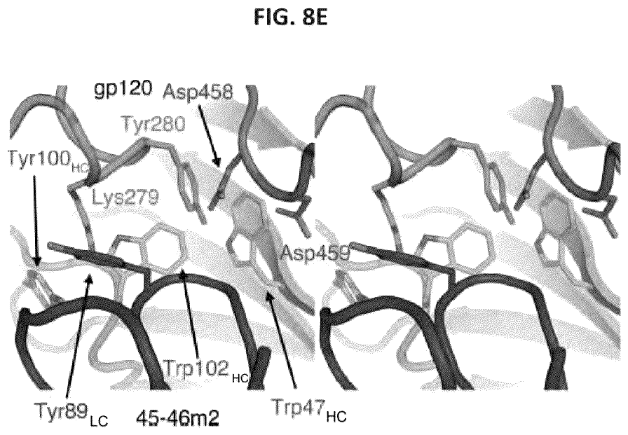

Considering the close contacts that 45-46m2 makes with Asn279_{gp120} (FIG. 8C), an N-linked glycan attached to Asn279gp120 would most likely prevent 45-46m2 binding to gp120s on spike trimers, resulting in resistance to neutralization. The saturation of the YU-2A28IT neutralization curves at values less than 100% (FIG. 11; FIG. 12C) is consistent with both a sensitive and a resistant population of virions, suggesting only partial incorporation of N-linked glycan attached to Asn279_{gp120}. Incomplete processing at the level of individual gp120 protomers would likely give rise to heterogeneously glycosylated trimeric Env spikes, i.e., trimers that were fully, partially, or not glycosylated at residue 279. At the population level, the sensitivity of YU-2A^{281T} viruses to 45-46m2-like antibodies would vary according to the Env composition of each virus, thereby giving rise to both resistant and sensitive virions within the set of YU-2^{A281T} viruses.

### Example 3. Combinations of Antibodies Improve Anti-HIV-1 Activity In Vitro and In Vivo

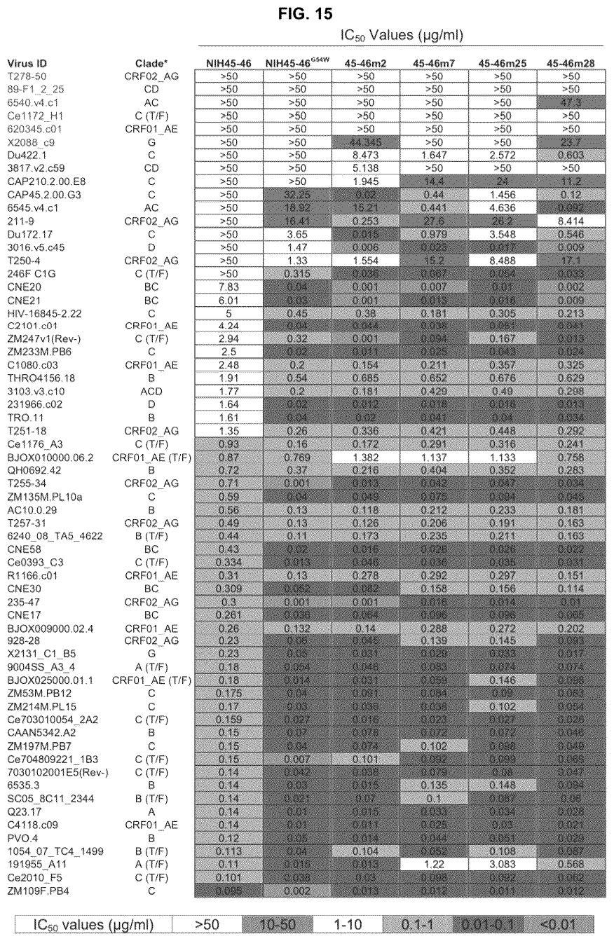

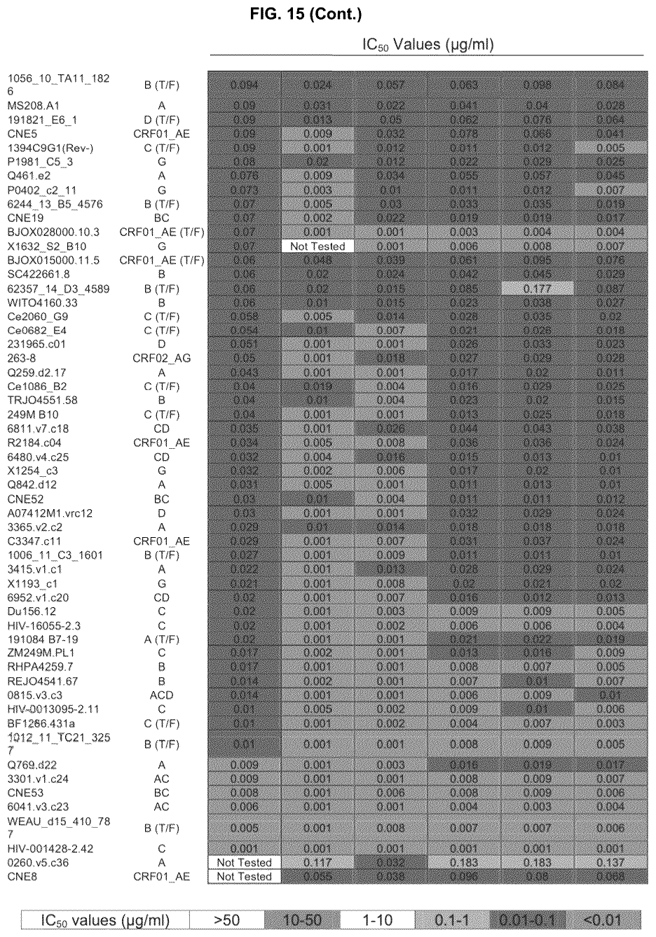

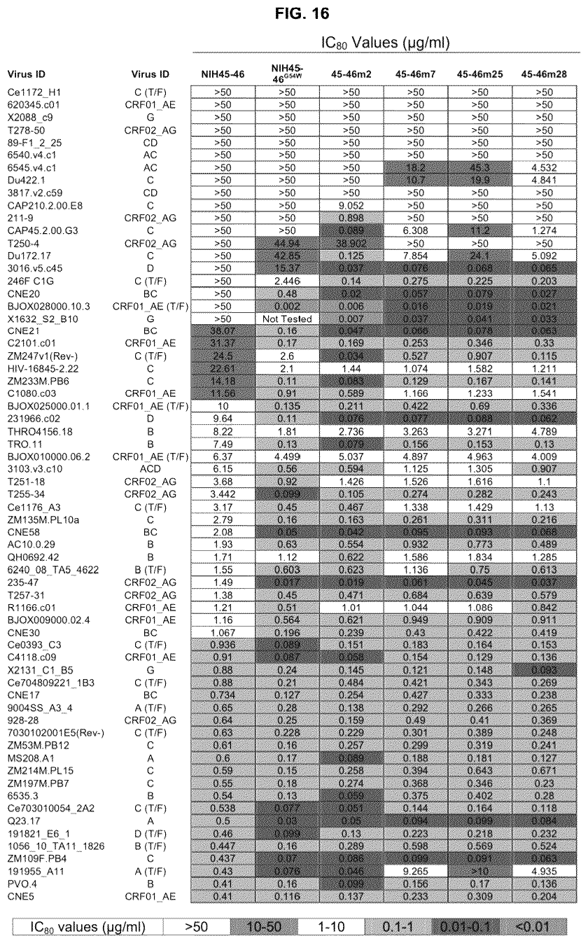

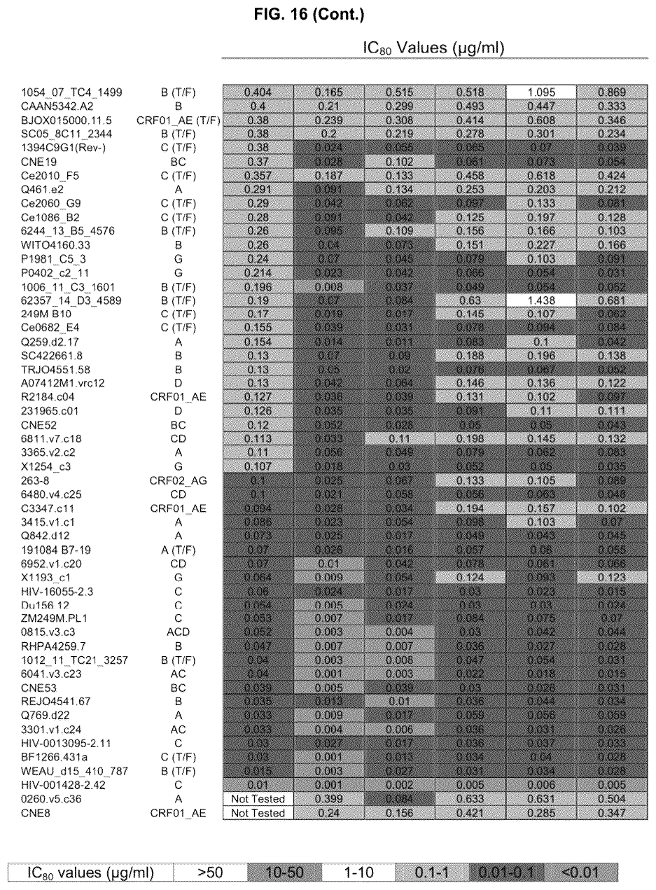

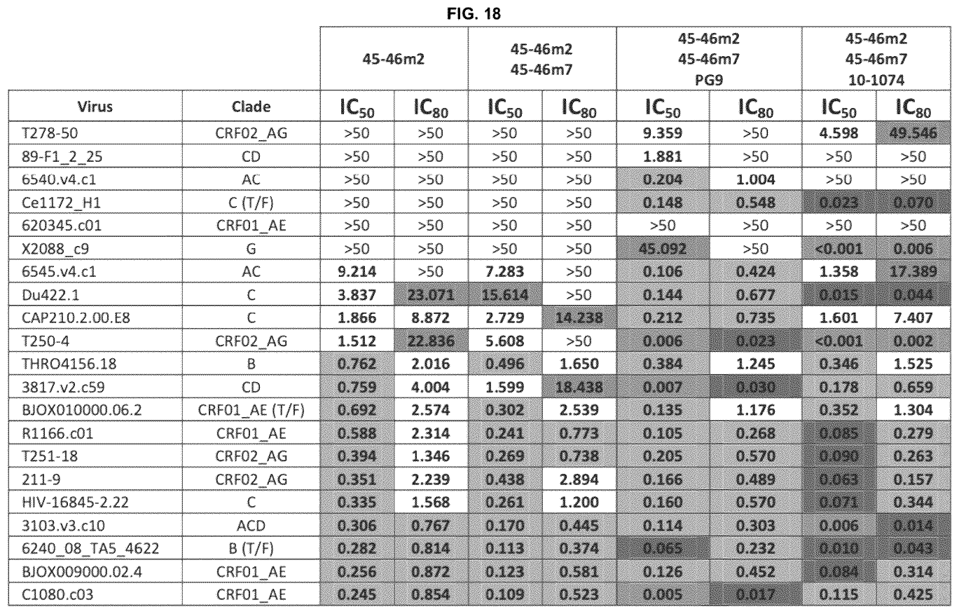

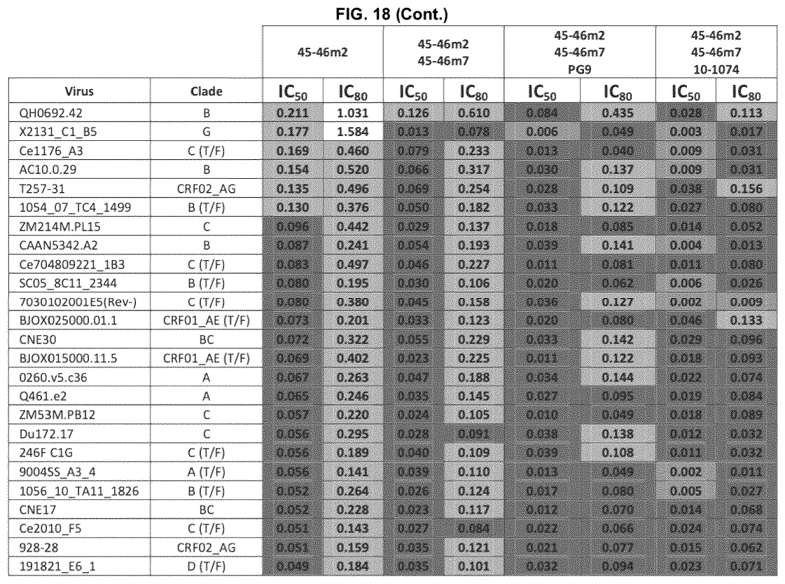

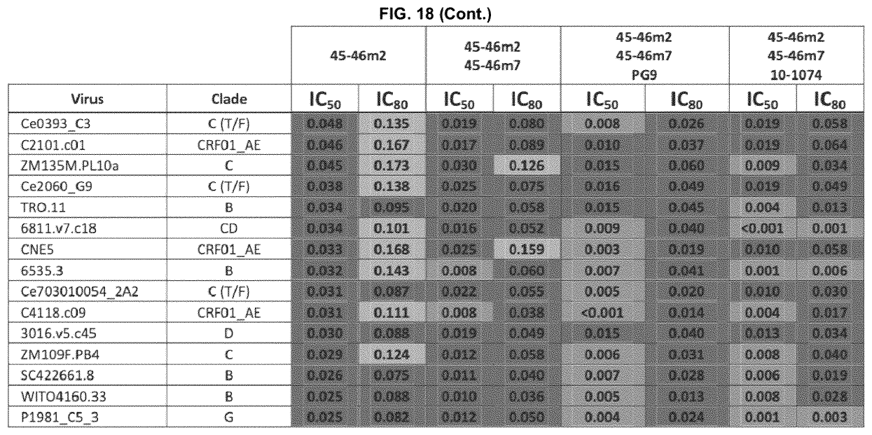

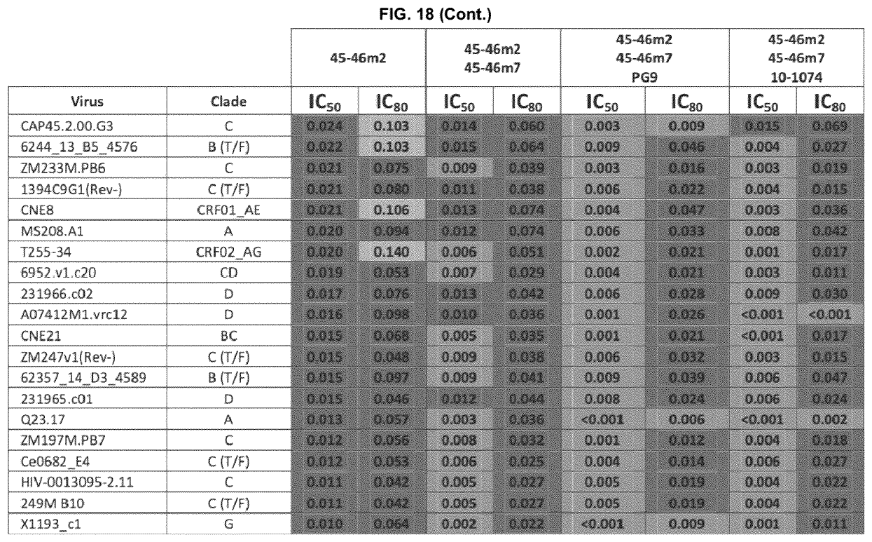

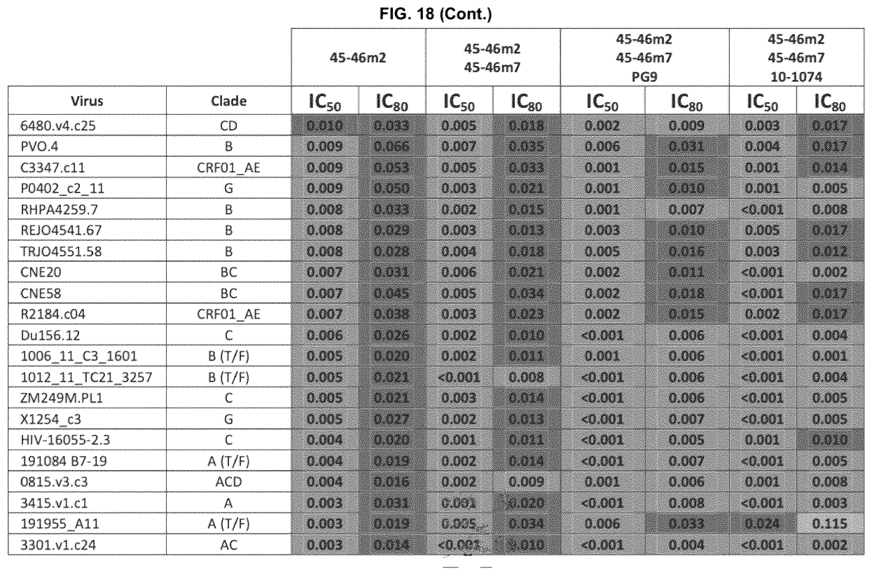

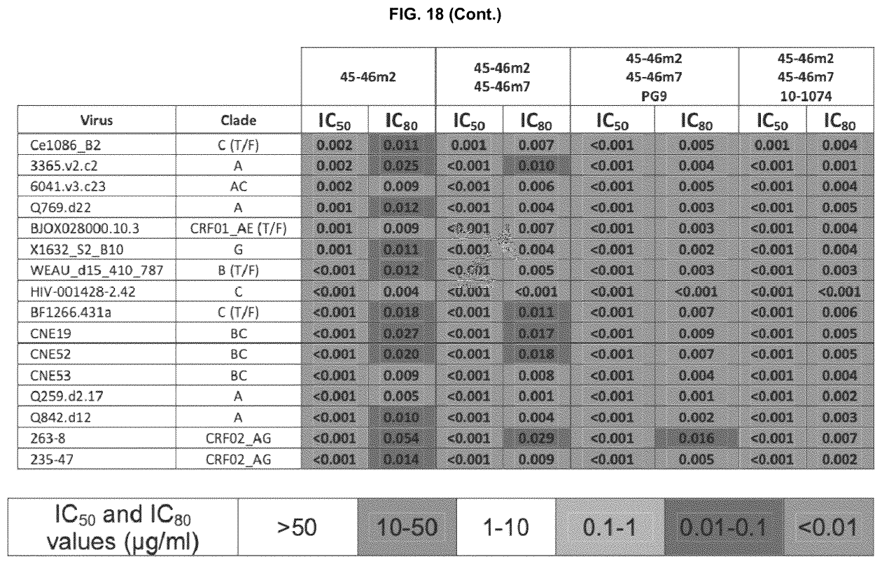

The breadth and potencies of selected antibodies were evaluated alone and in combination using the 118-strain cross-clade virus panel (FIGS. 15-16). 45-46m7, 45-46m25 and 45-46m28 effectively neutralized YU-2 N/DNGG consensus variants, but these antibodies and a 45-46m2/45-46m7 combination did not neutralize consensus variant strains that were resistant to 45-46m2 (FIGS. 15-16, 18). These results suggest that changing Trp47_{45-46m2(HC)} to a smaller amino acid can only partially alleviate steric constraints associated with PVL antibody binding to an N/DNGG consensus variant. Thus effective neutralization by 45-46m7, 45-46m25 and 45-46m28 of escape mutants that utilize non-N/DNGG consensus residues is likely only when the parent viral strain is sensitive to a PVL antibody. However, given the broad neutralization profiles of parental PVL antibodies (West et al., 2012, supra), strains resistant to parental PVLs and to 45-46m7, 45-46m25 and 45-46m28 due to changes in the gp120 N/DNGG consensus sequence are likely to be rare. Addition of 10-1074, a more potent clonal variant of PGT121 (Walker et al., 2011, Nature 477:466-470, the entire contents of which are herein incorporated by reference) that recognizes a carbohydrate-dependent epitope associated with the gp120 V3 loop (Mouquet et al., 2012, PNAS, 109: E3268-3277, the entire contents of which are herein incorporated by reference), or PG9, a carbohydrate-dependent bNAb recognizing a V1/V2 epitope (Walker et al., 2009, Science 326:285-289, the entire contents of which are herein incorporated by reference), into the mixture resulted in neutralization of almost all resistant strains (FIG. 18).

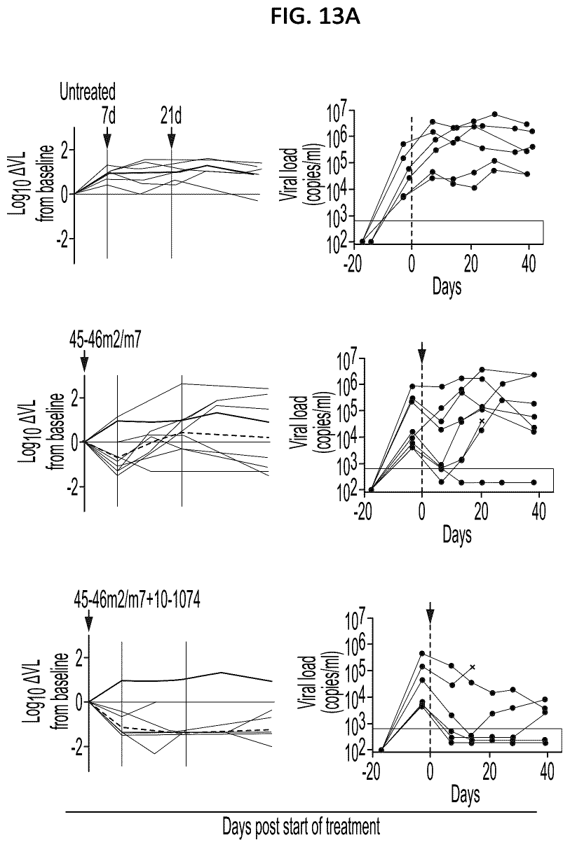

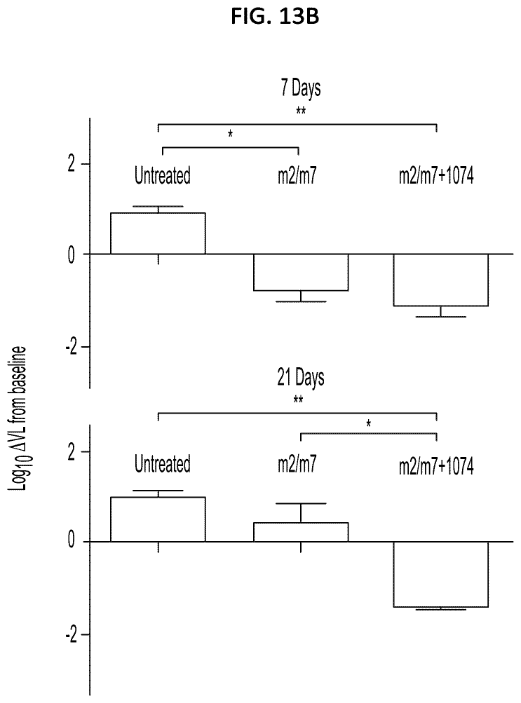

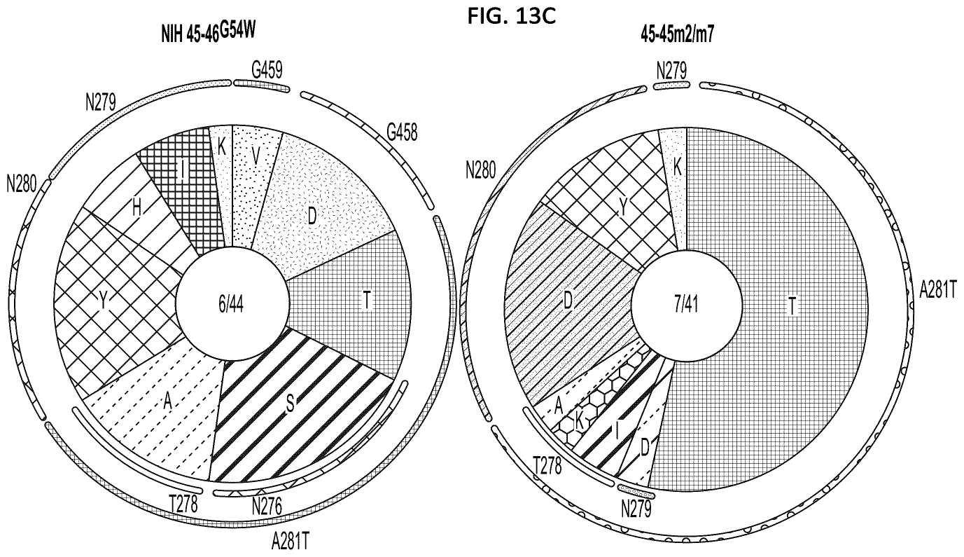

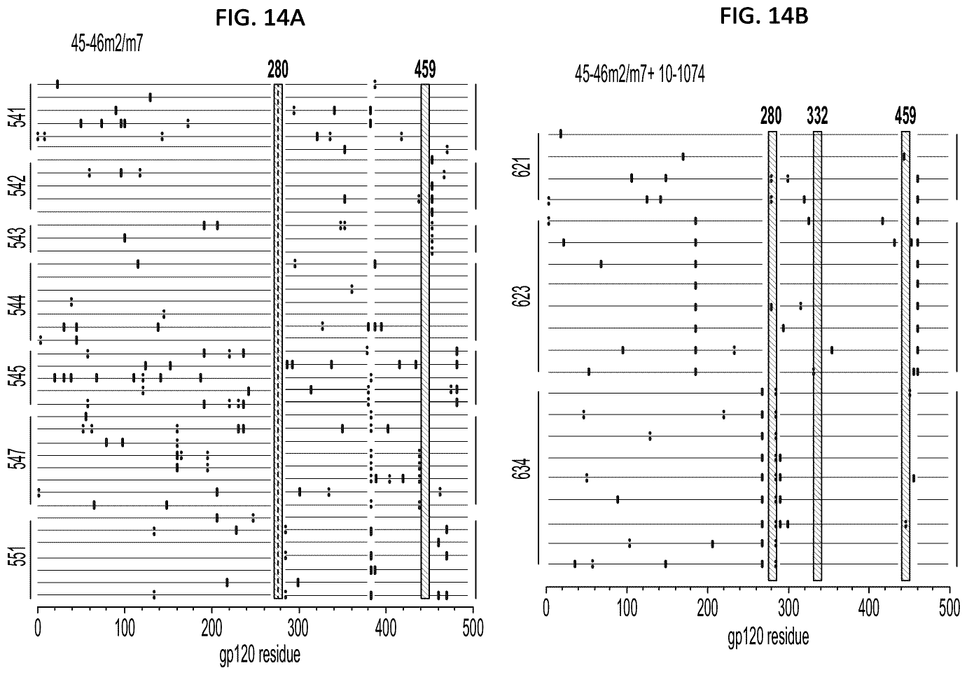

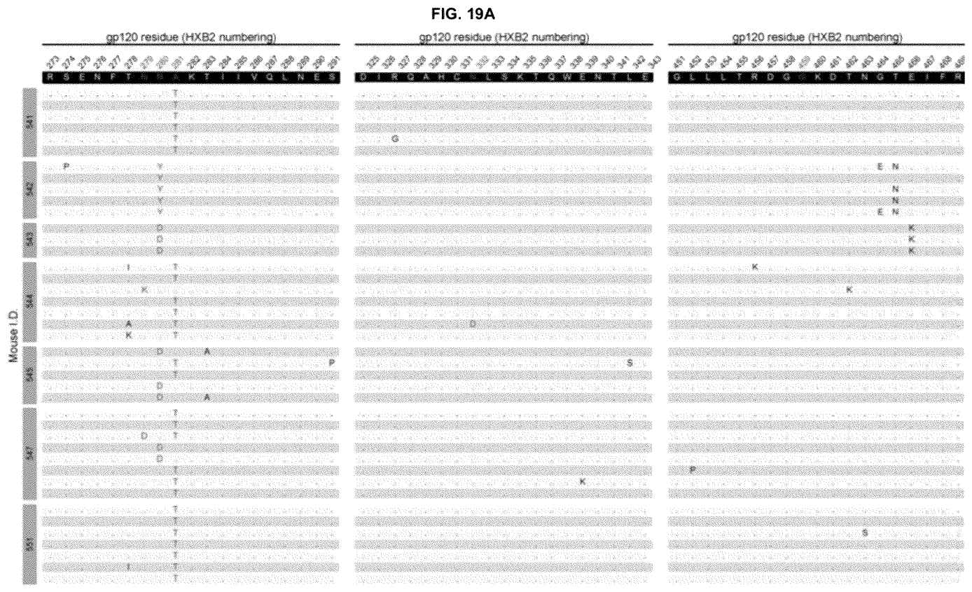

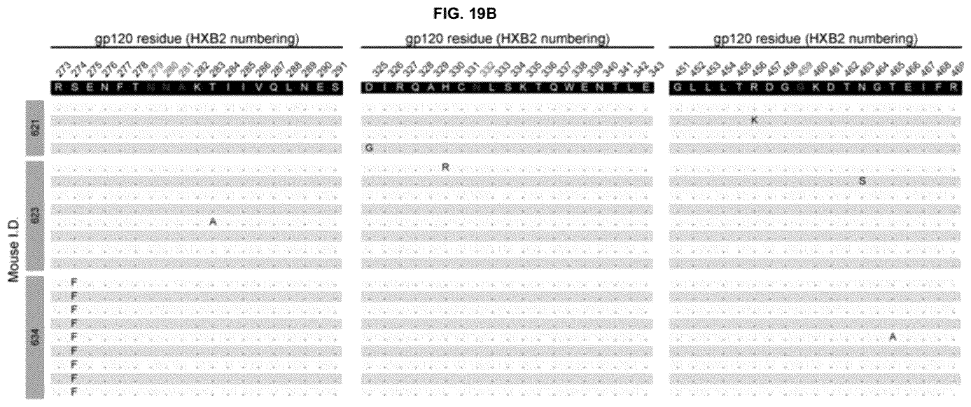

Antibodies can drive HIV-1 mutation or even control viral replication in humanized mice (Klein et al., 2012), offering the opportunity to examine HIV-1 escape mutations that arise in response to treatment with selected bNAbs. Escape mutations in HIV-1_{YU-2} that arose in response to a 45-46m2/45-46m7 combination were compared to monotherapy with NIH45-46^{G54W}.Treatment with 45-46m2/45-46m7 resulted in a significant initial drop in viremia by 7 days (FIGS. 13A-13B; p=0.0057). Although viremia rebounded to pretreatment levels after 21 days in seven of eight mice, the Env sequences isolated from the 45-46m2/45-46m7-treated viremic mice revealed striking differences compared with viruses isolated after escape from NIH45-46^{G54W} monotherapy (Klein et al., 2012) (FIG. 13C; FIG. 14A; FIG. 19A). Mutations in the GG portion of the N/DNGG consensus sequence (Gly458gp120 and Gly459_{gp120}), which resulted in resistance to NIH45-46^{G54W} (FIG. 10) and that were isolated following NIH45-46^{G54W} monotherapy (Klein et al., 2012), were absent (FIG. 13C; FIG. 14A; FIG. 19A). Although effective against potential mutations in the V5 region (residues 458_{gp120} and 459_{gp120}), the combination of 45-46m2 and 45-46m7 did not eliminate mutations in loop D (residues 279_{gp120} and 280_{gp120}). This may indicate that the antibody concentrations reached in vivo were not sufficient. Consistent with this suggestion, the in vitro IC50 values for 45-46m2 and 45-46m7 against loop D variants were >0.1 μg/ml whereas the IC50 values for V5 variants were <0.01 μg/ml (FIG. 10).

The predominant escape mutant found in viruses isolated from the 45-46m2/45-46m7-treated mice was A281T_{gp120}, a substitution that introduces a potential N-linked glycosylation site at Asn279_{gp120} and results in a less fit virus (FIG. 12B). In the context of an Asn279_{gp120}-linked glycan, compensatory mutations to remove the potential N-linked glycosylation site at Asn276_{gp120} were selected (FIG. 13C; FIG. 14A; FIG. 19A). Specifically, attachment of an N-linked glycan to Asn276_{gp120} was prevented by altering the asparagine (N276D and N276S) or the final residue (T278A) in the Asn-X-Ser/Thr potential N-linked glycosylation sequence motif. It is believed that a glycan attached to Asn279_{gp120} in a gp120 lacking Asn276_{gp120}-attached glycan could be pushed toward the empty space created by elimination of the Asn276_{gp120} glycan to facilitate binding to CD4. Thus, eliminating the glycan at Asn276_{gp120} could compensate for the otherwise unfavorable addition of a glycan to Asn279_{gp120}. The suggestion that mutations to remove an Asn276_{gp120}-linked glycan are compensatory mutations required when an Asn279_{gp120}-linked glycan is introduced rather than escape mutations on their own is consistent with potent neutralization of N276S and T278A mutants of YU-2 by NIH45-46^{G54W}, 45-46m2 and 45-46m7 (FIG. 12C) and the emergence of N276S and T278A mutations only when A281T was present (FIG. 14A; FIG. 19A).

When HIV-1_{YU2}-infected mice were treated with a combination of 45-46-m2, 45-46m7 and 10-1074 (Mouquet et al., 2012, PNAS, 109: E3268-3277, the entire contents of which are incorporated by reference), control of viremia in all animals that lived beyond 20 days after the start of treatment (FIGS. 13A-13B). With regards to the animal that died prior to this time, gp120 sequences just prior to its death did not harbor mutations that would indicate escape from either 10-1074 or 45-46m2/m7 (FIG. 14B; FIG. 19B). While some mice had detectable viral loads during treatment, known escape mutations were not found in viruses isolated during treatment for the bNAbs used in the treatment mix (FIG. 14B; FIG. 19B). Thus the combination of 45-46m2 and 45-46m7 effectively reduced the available pathways for escape, and the 45-46m2, 45-46m7 and 10-1074 combination potently treated HIV-1_{YU-2}-infected mice.

### Materials and Methods

### Example 4. Vector Construction, Protein Expression and Protein Purification

Modifications of NIH45-46 heavy and light chain genes were made using QuikChange Lightning (Agilent Technologies) and verified by DNA sequencing (Eton Bioscience). Antibodies were expressed as IgGs using described protocols (Diskin et al., 2010, Nature structural & molecular biology 17:608-613, the entire contents of which are herein incorporated by reference). Briefly, secreted IgGs from polyethyleneimine (25 kD, linear; Polysciences)-mediated, transiently-transfected HEK293-6E cells were captured on protein A or protein G affinity columns (GE Healthcare) and eluted in 100 mM citrate pH 3.0, 150 mM sodium chloride. Antibodies subsequently used in neutralization assays were dialyzed into 10 mM citrate pH 3.0, 150 mM sodium chloride and adjusted to a concentration of 1 mg/ml. Fab fragments for crystallization and binding assays were obtained by digesting IgGs in 20 mM Tris pH 8.0, 150 mM sodium chloride (TBS) with a 1:100 ratio of papain (Sigma) activated in 50 mM phosphate pH 7.0, 2 mM ethylenediaminetetraacetic acid, 10 mM cysteine at 37° C. until completion of the cleavage (20 min-60 min, monitored by SDS-PAGE). The Fc was removed by protein A chromatography and Fabs were further purified using Superdex 200 (GE Healthcare) 10/300 Size Exclusion Chromatography (SEC).

The clade A/E 93TH057-derived gp120 core (Zhou et al., 2010, Science 329:811-817, the entire contents of which are herein incorporated by reference) (a gp120 construct lacking the V1/V2 and V3 loops) was expressed in insect cells and purified using previously-described protocols (Diskin et al., 2011, supra). Briefly, supernatants from baculovirus-infected insect cells were collected, buffer exchanged into TBS and passed through a Ni²⁺-NTA affinity column (GE Healthcare). gp120 was eluted from the column using TBS plus 250 mM imidazole and purified using Superdex 200 16/60 SEC (GE Healthcare) in TBS supplemented with 0.02% (w/v) sodium azide.

### Example 5. In Vitro Neutralization Assays

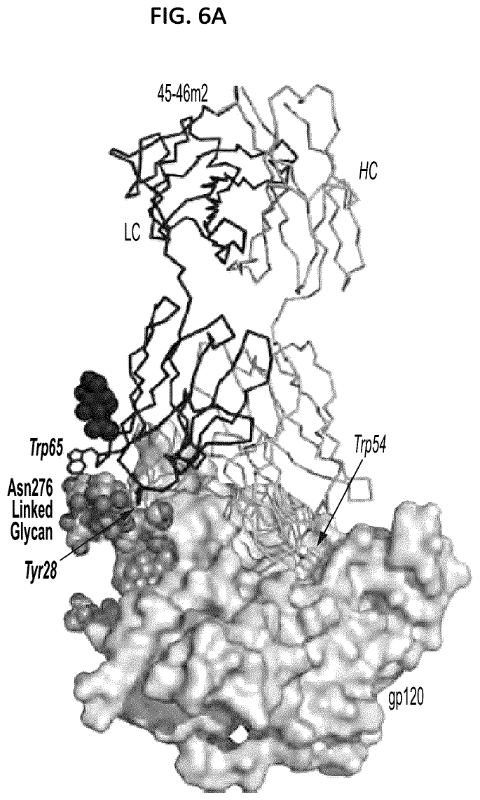

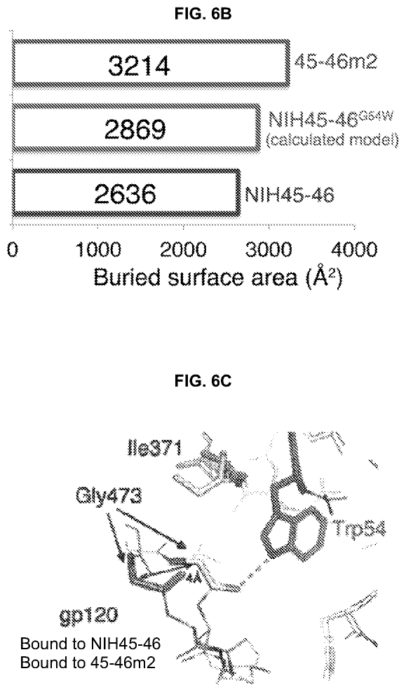

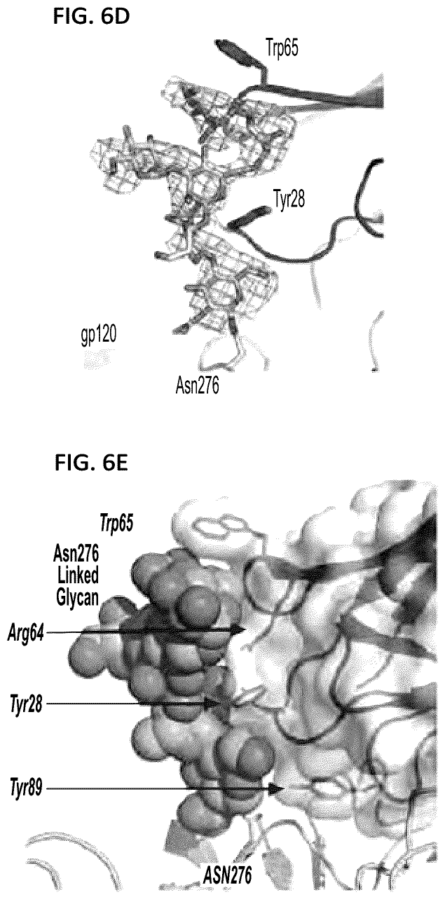

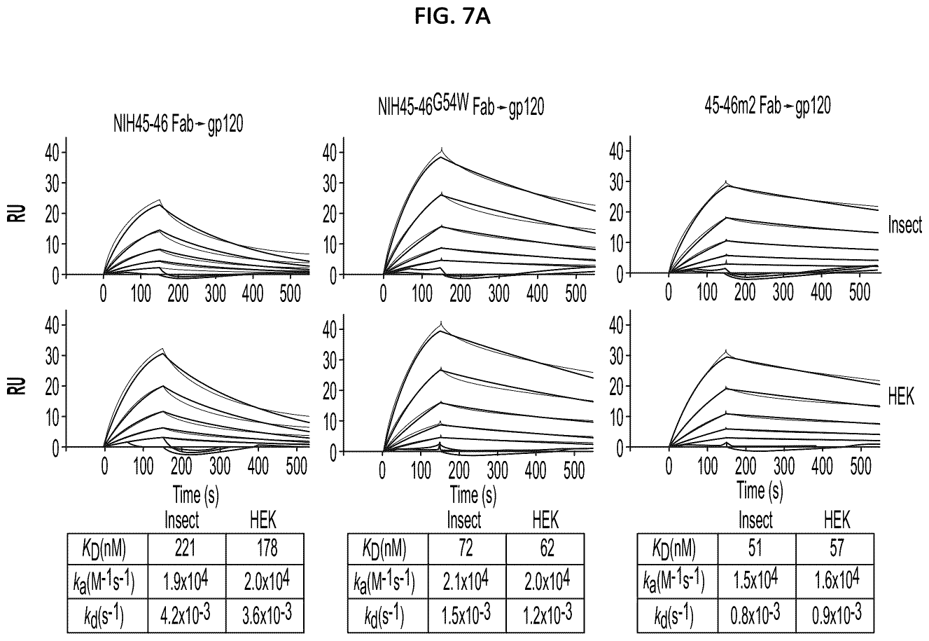

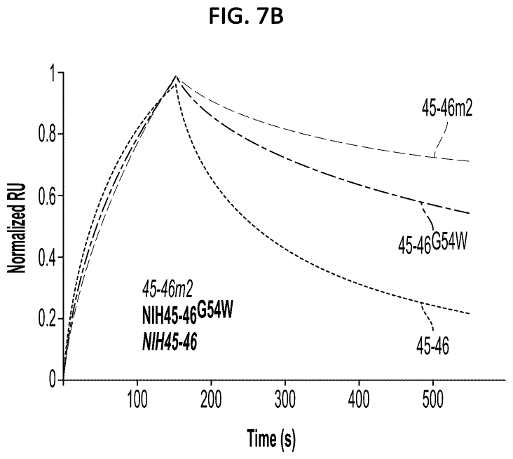

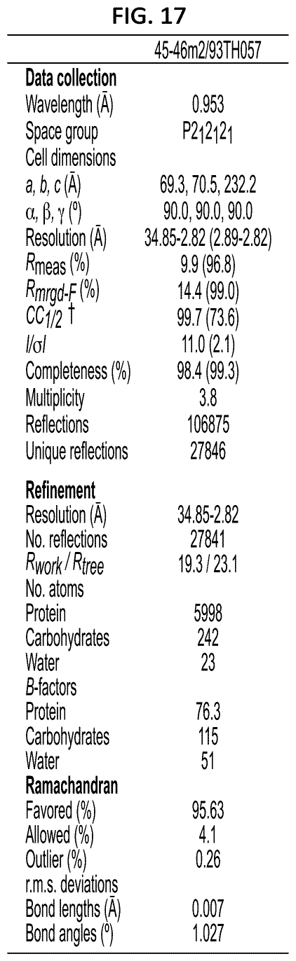

A previously-described pseudovirus neutralization assay was used Montefiori, 2005, Current protocols in immunology, Edited by John E. Coligan et al., Chapter 12, Unit 12.11, the entire contents of which are herein incorporated by reference) to assess the neutralization potencies of the various antibodies against multiple HIV-1 strains. YU-2 escape mutant pseudoviruses were generated by co-transfecting HEK293T cells with vectors encoding Env and a replication-deficient HIV-1 backbone as described (Montefiori, 2005). Neutralization assays were performed in-house for evaluating antibody mutants against the YU-2 escape mutants (FIG. 15; FIG. 9) and by the Collaboration for AIDS Vaccine Discovery (CAVD) core neutralization facility for testing a subset of the antibodies against a large panel of isolates (FIGS. 16-17). Some of the in-house data were derived from neutralization assays that were dispensed automatically by a Freedom EVO® (Tecan) liquid handler (IC₅₀ values derived from manual and robotic assays agreed to within 2-4 fold.) In all cases, neutralization was monitored by the reduction of a Tat-induced reporter gene (luciferase) in the presence of a three- or five-fold antibody dilution series (each concentration run in duplicate or triplicate) after a single round of pseudovirus infection in TZM-bl cell line (Montefiori, 2005). Antibodies were incubated with 250 viral infectious units at 37° C. for one hour prior to incubation with the reporter cells (10,000 per well) for 48 hours. Luciferase levels were measured from a cell lysate using BrightGlo (Promega) and a Victor3 luminometer (PerkinElmer). Data were fit by Prism (GraphPad) using nonlinear regression to find the concentration at which 50% inhibition occurred (IC₅₀ value). For evaluating the neutralization of YU-2 escape mutants, at least two independent experiments were performed. FIG. 6 lists the average IC₅₀ values for the various 45-46 mutants if 0.1< (IC₅₀ 1/IC₅₀ 2)<10. In cases where the two IC₅₀ values did not agree, additional experiments were performed. The reported IC₅₀ values for NIH45-46^{G54W}, 45-46m2, and 45-46m7 are averages calculated from at least five independent experiments.

### Example 6. Crystallization, Data Collection, Model Building and Refinement

45-46m2

Fab was purified by Superdex 200 (GE Healthcare) 10/300 SEC in 100 mM citrate pH 3.0, 150 mM sodium chloride and combined with an equimolar amount of 93TH057 gp120. After concentration using an Amicon™ (Millipore) spin column, the complex was incubated with 40,000 units of Endoglycosidase H (NEB) per 2 mg of gp120 in the absence of detergents at 37° C. for 16 hours in the manufacturer's recommended buffer. The complex was further purified using Superdex200 (GE Healthcare) SEC in TBS and concentrated to OD₂₈₀=9.5. Data for the structure determination were collected from rod-like crystals grown in a vapor diffusion sitting drop set at a final volume of 2 μl (1:1 protein/reservoir ratio) with 12% (v/v) isopropanol, 10% (w/v) polyethylene glycol 10,000 kD, 0.1 M citrate pH 5.0 at 20° C. The crystals were briefly soaked at 30% (v/v) isopropanol, 5% glycerol, 10% (w/v) polyethylene glycol 10,000 kD, 0.1 M citrate pH 5.0 before flash cooling using liquid nitrogen.

Data to 2.82 Å resolution were collected from a P21 21 21 45-46m2/gp120 complex crystal with similar cell dimensions as the NIH45-46/gp120 crystals (Diskin et al., 2011, supra) at the Stanford Synchrotron Radiation Lightsource (SSRL) beamline 12-2 using a Pilatus 6M (Dectris) detector and 0.9537 Å radiation (FIGS. 19A-19B). Data were indexed, integrated and scaled using XDS (Kabsch, 2010) (FIGS. 19A-19B). Using Phaser and the NIH45-46/gp120 complex (PDB: 3U7Y) as a search model, we found a molecular replacement solution comprising one 45-46m2 Fab and one gp120 in the asymmetric unit. Several rounds of simulated annealing were performed in initial refinement cycles to minimize model bias. The structure was refined using iterative cycles of refinement using the Phenix crystallography package and Coot for manual re-building. To facilitate refinement at 2.82 Å, the model was restrained using the NIH45-46/gp120 structure as a reference and applying secondary structure restraints. The final model (R_{free}=23.1%, R_{work}=19.3%) consists of 5998 protein atoms, 242 carbohydrate atoms and 23 water molecules. 95.63%, 4.1%, and 0.26% of the residues are in the favored, allowed, and disallowed regions, respectively, of the Ramachandran plot. The first glutamine of the 45-46m2 HC was modeled as 5-pyrrolidone-2-carboxylic acid. Disordered regions that were not modeled include residues 1-2 and 210 (the C-terminus) of the 45-46m2 LC, residues 133-136 and 219-221 (the C-terminus) of the 45-46m2 HC, and residues 302-308 (a short linker substituting for the V3 loop), residues 397-408 (a total of 6 residues from V4) and the 6x-His tag of 93TH057 gp120.

Structures were analyzed and figures were prepared using PyMol as described in Schrödinger, 2011, The PyMOL Molecular Graphics System, the entire contents of which are herein incorporated by reference). Buried surface areas were was calculated using a 1.4 Å probe using Areaimol as implemented in CCP4i package (Collaborative Computational Project Number 4, 1994).

### Example 7. Surface Plasmon Resonance (SPR) Measurements

SPR data were collected using a Biacore™ T200 instrument (GE Healthcare). Primary amine coupling chemistry was used to immobilize 1000 resonance units (RU) of the Fabs of NIH45-46, NIH45-46^{G54W}, or 45-46m2 in 10 mM acetate pH 5.0 at a concentration of 0.2 μM to a CM5 sensor chip as described in the Biacore™ manual. Flow channel 1 was mock coupled and served as a blank subtraction channel. gp120 protein was injected as a two-fold dilution series (500 nM to 31.2 nM) at a flow rate of 80 μL/min at 25° C. in 20 mM HEPES, pH 7.0, 150 mM sodium chloride and 0.005% (v/v) P20 surfactant, and sensor chips were regenerated using 10 mM glycine pH 2.5. A 1:1 binding model was fit to the blank-subtracted data using the Biacore™ analysis software to derive kinetic constants (kₐ and k_{d}; on- and off-rates) that were subsequently used to calculate affinities (KD; equilibrium dissociation constant).

### Example 8. In Vitro Viral Fitness Assays

Replication experiments were carried out as described previously (Neumann et al., 2005, Virology 333:251-262; Sather et al., 2012, J Virol 86:12676-12685, the entire contents of both of which are herein incorporated by reference) utilizing wild type YU-2 and three point mutants in gp120 designated as YU-^{2N279K}, YU-^{2N280D}, and YU-2^{A281T}. The entire gp160 portion of each env variant was inserted into the TN6 replication competent viral backbone, and each construct was transfected into 293T cells to produce infectious virions. Stimulated PBMCs were prepared from whole human blood by Ficoll gradient separation, followed by 72 hours of stimulation by culturing in complete RPMI containing 2 micrograms per ml IL-2 and 3 μg/mL phytohemagglutinin (PHA). 15×10⁶ stimulated PBMCs were infected for 3 hours with viral inoculum containing the equivalent of 12.5 pg of HIV p24. After inoculation, the cells were re-suspended in fresh complete RPMI/IL-2 media at a density of 3×10⁶ cells per ml. At 2-3 day intervals, half of the culture supernatant was harvested and replaced with fresh media. Harvested supernatants were assayed for p24 content by capture ELISA (Zeptometrix, Buffalo, N.Y.). During the culture period, the cultures were monitored to ensure that viability remained above 90%.

### Example 9. In Vivo Therapy Experiments

HIV-1 escape experiments were performed in HIV-1_{YU2}-infected humanized mice as previously described in Klein et al., 2012, Nature 492:118-122, the entire contents of which are herein incorporated by reference). Briefly, non-obese diabetic Rag1^{-/-} IL2Rγ^{NULL}. (NRG) mice (Jackson Laboratory, Bar Harbor, Me.) were reconstituted with fetal liver-derived hematopoietic stem cells and infected with HIV-1_{YU2} (57.5 ng p24). Mice with viral loads >4×10³ copies/ml at 14-17 days post infection were included in treatment experiments. Antibody-treated mice were injected subcutaneously with 1.5 mg 45-46m2 and 1.5 mg 45-46m7 every two days, and mice receiving 10-1074 were injected with 0.5 mg antibody twice per week. All experiments were performed with authorization from the Institutional Review Board and the IACUC at the Rockefeller University.

### Example 10. Viral Load Measurements and Sequence Analysis

Viral load and sequence analysis of HIV-1 gp120 were performed as previously described (Klein et al., 2012, supra).

Briefly, total RNA was extracted from 100 μl EDTA-plasma using the QiaAmp MinElute Virus Spin Kit as per the manufacturer's protocol. Viral RNA was detected by quantitative reverse-transcriptase PCR using a Stratagene Mx3005P real-time thermal cycler. HIV-specific forward and reverse primer sequences were 5′-GCCTCAATAAAGCTTGCCTTGA-3′ (SEQ ID NO: 47) and 5′-GGCGCCACTGCTAGAGATTTT-3′ (SEQ ID NO: 48), respectively. An internal probe (5′-AAGTAGTGTGTGCCCGTCTGTTRTKTGACT-3′) (SEQ ID NO: 49) contained a 5′ 6-carboxyfluorescein reporter and internal/3′ ZEN-Iowa Black® FQ double-quencher (Integrated DNA Technologies, Inc., Coralville, Iowa). The reaction mix was prepared using the TaqMan® RNA-to-Ct™ 1-Step kit (Applied Biosystems, Foster City, Calif.). Cycle threshold values were converted to viral loads using an HIV-1 (NL4/3-YU-2) viral preparation of known copy number as a standard.

For gp120 sequencing, viral cDNA was generated from extracted viral RNA (described above) using Superscript III Reverse Transcriptase (Invitrogen) and amplified by gp120-specific nested PCR using the Expand Long Template PCR System (Roche). PCR amplicons were gel purified, cloned into pCR4-TOPO® (Invitrogen), transformed into One-Shot TOP10® cells (Invitrogen) and sequenced using the insert-flanking primers M13F and M13R. Sequence reads were assembled using Gencious Pro software version 5.5.6 (Biomatters Ltd) and aligned to HIV-1^{YU2} gp120 (accession number M93258). Manual edits to sequence assemblies and alignments were performed in Geneious. gp120 residues were numbered according to HXB2, as determined by the Los Alamos Sequence Locator tool.

As disclosed throughout, a PVL antibody such as NIH45-46 having three substitutions as described herein, results in a potent antibody that is capable of neutralizing a broad range of HIV viral strains. Furthermore, this triple mutant antibody in combination with a second select antibody further increases its potency.

While the present invention has been illustrated and described with reference to certain exemplary embodiments, those of ordinary skill in the art will understand that various modifications and changes may be made to the described embodiments without departing from the spirit and scope of the present invention, as defined in the following claims.

SEQUENCE LISTING SEQ ID NOS: 1-46

| | Light Chain | Heavy Chain |
|---|---|---|
| Antibody Name | SEQ ID NO: | SEQ ID NO: |
| VRC01 | 1 | 2 |
| | EIVLTQSPGTLSLSPGETAIISCRTSQYGSLAWYQ | QVQLVQSG-- |
| | QRPGQAPRLVIYSGSTRAAGIP | GQMKKPGESMRISC |
| | DRFSGSRWGPDYNLTISNLESGDFGVYYCQQYE | RASG---YEFI------ |
| | FFGQGTKVQVDIKR | DCTLNWIRLAPGKR |
| | | PEWMGWLKPRGGA |
| | | VNYARPLQGRVTM |
| | | TRDVYSDTAFLELR |
| | | SLTVDDTAVYFCTR |
| | | GKNCDYNWDFEHW |
| | | GRGTPVIVSS |
| VRC02 | 3 | 4 |
| | EIVLTQSPGTLSLSPGETAIISCRTSQYGSLAWYQ | QVQLVQSGGQMKK |
| | QRPGQAPRLVIYSGSTRAAGIPDRFSGSRWGPDY | PGESMRISCQASGYE |
| | NLTIRNLESGDFGLYYCQQYEFFGQGTKVQVDI | FIDCTLNWVRLAPG |
| | KR | RR |
| | | PEWMGWLKPRGGA |
| | | VNYARPLQGRVTM |
| | | TRDVYSDTAFLELR |
| | | SLTADDTAVYYCTR |
| | | GKNCDYNWDFEHW |
| | | GRGTPVTVSS |
| NIH-45-46 | 5 | 6 |
| | EIVLTQSPATLSLSPGETAIISCRTSQSGSLAWYQ | QVRLSQSG-- |
| | QRPGQAPRLVIYSGSTRAAGIP | GQMKKPGESMRLSC |
| | DRFSGSRWGADYNLSISNLESGDFGVYYCQQYE | RASG--YEFL------ |
| | FFGQGTKVQVDIKRTVA | NCPINWIRLAPGRRP |
| | | EWMG |
| | | WLKPRGGAVNY- |
| | | ARKFQGRVTMTRD |
| | | VY---- |
| | | SDTAFLELRSLTSDD |
| | | TAVYFCTRGKYCTA |
| | | RDYYNWDFEHWGR |
| | | GAPVTVSS |
| 3BNC60 | 7 | 8 |
| | DIQMTQSPSSLSARVGDTVTITCQANGYLNWYQ | QVHLSQSG-- |
| | QRRGKAPKLLIYDGSKLERGVP | AAVTKPGASVRVSC |
| | ARFSGRRWGQEYNLTINNLQPEDVATYFCQVYE | EASG--YKIS------ |
| | FIVPGTRLDLKRTVA | DHFIHWWRQAPGQ |
| | | GLQWVG |
| | | WINPKTGQPNN- |
| | | PRQFQGRVSLTRQA |
| | | SWDFDTYSFYMDLK |
| | | AVRSDDTAIYFCAR |
| | | QRS |
| | | DFWDFDVWGSGTQ |
| | | VTVSS |

What is claimed is:

1. A nucleic acid molecule encoding an isolated anti-CD4 binding site (anti-CD4bs) VRC01-related antibody variant having a heavy chain and a light chain, comprising a heavy chain variable region and a light chain variable region,
the heavy chain variable region comprising:
a first substitution at position 54 of the heavy chain variable region having the amino acid sequence of SEQ ID NO: 85 according to Kabat numbering, wherein the first substitution is selected from the group consisting of glycine, histidine, arginine, glutamine, asparagine, lysine, glutamic acid, and aspartic acid, and
a second substitution at position 47 of the heavy chain variable region having an amino acid sequence of SEQ ID NO: 85 according to Kabat numbering, wherein the second substitution is selected from valine, isoleucine, and threonine; and
the light chain variable region comprising:
a light chain substitution for serine at position 28 of the light chain variable region having the amino acid sequence of SEQ ID NO: 84 according to Kabat numbering.

2. The nucleic acid molecule of claim 1, wherein the first substitution is arginine.

3. The nucleic acid molecule of claim 1, wherein the anti-CD4bs VRC01-related antibody variant is 45-46m2.

4. A vector comprising the nucleic acid molecule of claim 1.

5. A cell comprising the vector of claim 4.
